(19) United States
(12) Patent Application Publication
(10) Pub. No.: US 2006/0263294 A1
(43) Pub. Date: Nov. 23, 2006

(54) Tumor targeting agents and uses thereof

(75) Inventors: Mathias Bergman, Ostersundom (FI); Merja Auvinen, Espoo (FI); Hannu Elo, Helsinki (FI)

Correspondence Address: BUCHANAN, INGERSOLL & ROONEY PC, POST OFFICE BOX 1404, ALEXANDRIA, VA, 22313-1404, US

(73) Assignee: KARYON OY, Helsinki (FI)

(21) Appl. No.: 10/530,016

(22) PCT Filed: Oct. 3, 2003
(86) PCT No.: PCT/FI03/00724

§ 371 Date: Apr. 1, 2005

(30) Foreign Application Priority Data
Oct. 3, 2002 (FI) 20021761

(51) Int. Cl. A61K 51/00; A61K 49/10; C07K 7/08; C07K 7/06; C07K 9/00

(52) U.S. Cl. 424/1.69; 514/8; 514/15; 514/16; 514/17; 424/9.34; 530/322; 530/329; 530/330

(57) ABSTRACT

This invention relates to novel tumor targeting motifs, units and agents, as well as tumor targeting peptides and ana-logues thereof. The targeting agents typically comprise at least one targeting motif, Aa-Bb-Cc, and at least one ef-fector unic. The invention further relates to specific tumor targeting peptides, pharmaceutical and diagnostic com-posisitons comprising such peptides. Disclosed are also methods for diagnosing or treating cancer.

### FIELD OF THE INVENTION

The present invention relates to tumor targeting agents comprising at least one targeting unit and at least one effector unit, as well as to tumor targeting units and motifs. Further, the present invention concerns pharmaceutical and diagnostic compositions comprising such targeting agents or targeting units, and the use of such targeting agents and targeting units as pharmaceuticals or as diagnostic tools. The invention further relates to the use of such targeting agents and targeting units for the preparation of pharmaceutical or diagnostic compositions and for the preparation of reagents to be used in diagnosis or research. Furthermore, the invention relates to kits for diagnosing or treating cancer and metastases. Still further, the invention relates to methods of removing, selecting, sorting and enriching cells, and to materials and kits for use in such methods.

### BACKGROUND OF THE INVENTION

Malignant tumors are one of the greatest health problems of man as well as animals, being one of the most common causes of death, also among young individuals. Available methods of treatment of cancer are quite limited, in spite of intensive research efforts during several decades. Although curative treatment (usually surgery in combination with chemothreapy and/or radiotherapy) is sometimes possible, malignant tumors (cancer) still are one of the most feared diseases of mankind, requiring a huge number of lives every year. In fact, curative treatment is rarely accomplished if the disease is not diagnosed early. In addition, certain tumor types can rarely, if ever, be treated curatively.

There are various reasons for this very undesirable situation but the most important one is clearly the fact that nearly all (if not all) treatment schedules (except surgery) lack sufficient selectivity. Chemotherapeutic agents commonly used, such as alkylating agents, platinum compounds (e.g. cisplatin), bleomycin-type agents, other alkaloids and other cytostatic agents in general, do not act on the malignant cells of the tumors alone but are highly toxic to other cells as well, being usually especially toxic to rapidly dividing cell types, such as hematopoietic and epithelial cells. The same applies to radiotherapy.

In addition to the above mentioned complications, two further major problems plague the non-surgical treatment of malignant solid tumors. First, physiological barriers within tumors impede the delivery of therapeutics at effective concentrations to all cancer cells. Second, acquired drug resistance resulting from genetic and epigenetic mechanisms reduces the effectiveness of available drugs.

The treatment of cancer patients with currently available, largely non-selective, chemotherapeutic agents or radiotherapy results often also in undesirable side effects. In order to improve the effect of chemotherapeutic agents and to diminish the side effects it would be extremely important to identify agents that are capable of targeting to specific organs or tissues or to tumor tissues and to carry the desired cytotoxic or other drugs specifically to these organs or tissues.

The same applies also to a specific field of cancer treatment, namely neutron capture therapy, in which a non-radioactive nucleus (e.g. ¹⁰B, ¹⁵⁷Gd or ⁶Li) is converted into a radioactive nucleus in vivo in the patient with the aid of thermal (slow) neutrons from an external source. In this case, some prior art agents are claimed to have some 2-3 fold selectivity for at least some types of tumors, but the results obtained have been mainly disappointing and negative. Specific targeting agents would offer remarkable advantages also in this field.

Also in the diagnosis of cancer and of metastases, including the follow-up of patients and the study of the effects of treatment on tumors and metastases, more reliable, more sensitive and more selective methods and agents would be a great advantage. This is true for all methods currently in use, such as nuclear magnetic resonance imaging (NMR, MRI), X-ray methods, histological staining methods (for light microscopy and electron microscopy and related methods, and in the future possibly also NMR, infrared, electron spin resonance and related methods) and in general any imaging as well as laboratory methods (histology, cytology, cell sorting, hematological studies, FACS and so on) known by specialists in the field. Here, agents capable of targeting an entity for detection (a spin label, a radioactive substance, a paramagnetic contrast agent for NMR or a contrast agent for X-ray imaging or tomography, a boron atom for neutron capture and so on) specifically or selectively to tumor tissues, metastases or tumor cells and/or to tumor endothelium would be a great advantage.

Solid tumor growth is angiogenesis-dependent, and a tumor must continuously stimulate the growth of new microcapillaries for continued growth. Tumor blood vessels are structurally and functionally different from their normal resting counterparts. In particular, endothelial cells lining new blood vessels are abnormal in shape, they grow on top of each other and project into the lumen of the vessels. This neovascular heterogeneity depends on the tumor type and on the host organ in which the tumor is growing. Therefore vascular permeability and angiogenesisis are unique in every different organ and in tumor tissue derived from the organ.

There are numerous publications disclosing peptides homing to different cell and tissue types. Some of these are claimed to be useful as cancer targeting peptides. Among the earliest identified homing peptides described are the integrin and NGR-receptor targeting peptides described by Ruoslahti et al., in e.g., U.S. Pat. No. 6,180,084. These peptides home to angiogenic vasculature and bind to the NGR-receptor.

When tumors switch to the angiogenic phenotype and recruit new blood vessels, endothelial cells in these vessels express proteins on the luminal surface that are not produced by normal quiescent vascular endothelium. One such protein is αvβ3 integrin. US Patent publication, U.S. Pat. No. 6,177,542, discloses a peptide that can bind specifically to αvβ3 integrin. The tumor vessel specific targets described are adhesion molecules that mediate binding of endothelial cells to the vascular basement membrane. This peptide is a nine-residue cyclic peptide containing an ArgGlyAsp (RGD) sequence. Pasqualini et al., (1997) showed that when injected intravenously the peptide was able to home to blood vessels of murine and human tumors in mice 40-80 fold more efficiently than to those of control organs. It was suggested that RGD peptides may be suitable tools in tumor targeting for diagnostic and therapeutic purposes. However, integrin-binding peptides may interfere with cell attachment in general, and are thus not suitable for clinical applications for selective tumor targeting.

International Patent Publication WO 00/67771 provides endostatin peptides comprising the amino acid sequence RLQD, RAD, DGK/R. Other examples of peptides that home to angiogenic vasculature are described in U.S. Pat. Nos. 5,817,750 and 5,955,572. These peptides recognize RGD.

U.S. Pat. No. 5,628,979 describes oligopeptides for in vivo tumor imaging and therapy. The oligopeptides contain 4 to 50 amino acids, which contain as a characteristic triplet the amino acid sequence Leu-Asp-Val (LDV). This triplet is reported to provide the oligopeptide with in vivo binding affinity for LDV binding sites on tumors and other tissues.

International Patent publication WO 99/47550 describes cyclic peptides, containing an HWGF motif, that are specific inhibitors of MMP-2 and MMP-9. They have also found that the cyclic decapeptide CTTHWGFTLC specifically inhibits the activities of these enzymes, suppresses migration of both tumor cells and endothelial cells in vitro, homes to tumor vasculature in vivo, and prevents the growth and invasion of tumors in mice. However, peptides that act as inhibitors of MMPs show background binding to non-tumor tissues. The fact that MMPs are expressed also in normal tissue throughout the body also makes the administration of such peptides to humans or animals hazardous and even fatal, since the activity of these enzymes is required for normal tissue functions (Hidalgo and Eckhardt, 2001).

US Patent publication US 2002/0102265A1 describes a peptide, TSPLNIHNGQKL, that targets squamous cell cancer cell lines, and becomes internalized into cells in vitro. This peptide also targets experimental squamous carcinomas in nude mice.

U.S. Pat. Nos. 5,622,699 and 6,068,829 disclose a family of peptides comprising an SRL motif, which selectively home to brain.

International Patent publication WO 02/20769 discloses methods for identifying tissue specific peptides by phage display and biopanning. Some of the identified peptides are suggested to be tumor specific.

Although there are known homing peptides that bind to tumor vasculature, there are still very scarce reports on targeting agents that actually target tumor cells and tissues in vivo. Most of the previously described targeting peptides are vasculature specific. Thus, there is still an established need for new agents that target selectively to tumor tissue, tumor vasculature, or both.

For therapeutic applications, targeting peptides have been conjugated to doxorubicin in an uncontrolled fashion, obviously resulting in mixtures of products or at least in an undefined structure and possibly also resulting in unefficient action and especially in difficulties in the identification, purification, quality control and quantitative analysis of the agent, even the amount of doxorubicin per peptide molecule remaining unknown (e.g. Arap et al., 1998). The unspecific conjugation process might also impair the targeting functions of the peptide.

Another very serious disadvantage of the prior art is that most of the described targeting peptides appear to target to the tumor endothelium only and not to the tumor mass itself. For example, the targeting peptide used by Nicklin et al. (2000) directed adenovirus DNA transfection to resting endothelial cells in vitro, under conditions that hardly could be applied in vivo.

The targeting units according to the present invention offer an advantage over the prior art in that they seem to target to both the tumor endothelium and the tumor cell mass. This fact provides the possibility to target and destroy tumor endothelium supporting tumor growth as well as the tumor mass itself. A major advantage of this approach comes from the fact that the endothelium is a genetically stable tissue that will not acquire drug resistance but will be irreversibly eliminated.

It is not known whether the prior art targeting peptides are universal in the sense of being capable to target to any malignant tumor type. Thus, their use as targeting therapeutic agents to a certain specified tumor may be completely useless, giving no therapeutic advantage or effect over the free therapeutic agent itself. An even more serious drawback is that the use of such targeting agents in diagnostic procedures may not reveal all existing tumors and the malignant process may remain unrecognized.

The present invention offers a significant improvement in view of the prior art, since the targeting agents here described were found to target to all of the various tumor types tested. Remarkably, they target, for example, sarcomas, such as Kaposi's sarcoma, ornithine decarboxylase (ODC) overexpressing, highly angiogenic tumors, carcinomas, and to human primary and metastatic melanomas.

### BRIEF DESCRIPTION OF THE INVENTION

It is an object of the present invention to provide novel tumor and angiogenic tissue targeting agents that comprise at least one targeting unit and, optionally, at least one effector unit. In particular, the invention provides targeting units comprising at least one motif that is capable of targeting both tumor endothelium and tumor cell mass. Such targeting units, optionally coupled to at least one effector unit, are therapeutically and diagnostically useful, especially in the treatment and diagnosis of cancer, including metastases. Furthermore the targeting agents according to the present invention are useful for cell removal, selection, sorting and enrichment.

It is a second object of this invention to provide pharmaceutical and diagnostic compositions comprising at least one targeting agent or at least one targeting unit comprising at least one motif capable of specifically targeting tumors, tumor cells and tumor endothelium.

Further, it is a third object of the invention to provide novel diagnostic and therapeutic methods and kits for the treatment and/or diagnosis of cancer.

The present invention is based on the finding that a group of peptides having specific amino acid sequences or motifs are capable of selectively targeting tumors in vivo and tumor cells in vitro. Thus, the peptides of this invention, when administered to a human or animal subject, are capable of selectively binding to tumors but not to normal tissue in the body.

The present invention is also directed to the use of the targeting agents and analogues thereof for the manufacture of a pharmaceutical or diagnostic composition for treating or diagnosing cancer.

The targeting units of this invention may be used as such or coupled to at least one effector unit. Such substances can destroy the tumors or hinder their growth. The targeting units and targeting agents of this invention can target also metastases and therefore they may be used to destroy or hinder the growth of metastases. As early diagnosis of metastases is very important for successful treatment of cancer, an important use of the targeting units and targeting agents of this invention is in early diagnosis of tumor metastases.

The present invention further encompasses salts, derivatives and analogues of the targeting units and targeting agents, as described herein, as well as uses thereof.

It is a further object of the present invention to provide diagnostic and pharmaceutical compositions comprising targeting agents according to the present invention, as well as therapeutic and diagnostic methods for the treatment and diagnosis of cancer, utilizing targeting agents according to the present invention. Also provided are kits for use in such methods or for research purposes, as well as in cell sorting or removal.

Especially preferred embodiments of the present invention relate to a group of small, cyclic tumor targeting peptides comprising a motif, LRS or SRL, optionally coupled to an effector unit and other additional units, as described in more detail herein.

### BRIEF DESCRIPTION OF THE DRAWINGS

FIG. 1 is a graph showing the therapeutic effect of a targeting agent comprising doxorubicin.

### DETAILED DESCRIPTION OF THE INVENTION

For the purpose of this invention, the term “cancer” is used herein in its broadest sense, and includes any disease or condition involving transformed or malignant cells. In the art, cancers are classified into five major categories, according to their tissue origin (histological type): carcinomas, sarcomas, myleomas, and lymphomas, which are solid tumor type cancers, and leukemias, which are “liquid cancers”. The term cancer, as used in the present invention, is intended to primarily include all types of diseases characterized by solid tumors, including disease states where there is no detectable solid tumor or where malignant or transformed cells, “cancer cells”, appear as diffuse infiltrates or sporadically among other cells in healthy tissue.

The terms “amino acid” and “amino alcohol” are to be interpreted herein to include also diamino, triamino, oligoamino and polyamino acids and alcohols; dicarboxyl, tricarboxyl, oligocarboxyl and polycarboxylamino acids; dihydroxyl, trihydroxyl, oligohydroxyl and polyhydroxylamino alcohols; and analogous compounds comprising more than one carboxyl group or hydroxyl group and one or more amino groups.

By the term “peptide” is meant, according to established terminology, a chain of amino acids (peptide units) linked together by peptide bonds to form an amino acid chain. Peptides may be cyclic as described below. For the purposes of the present invention, also compounds comprising one or more D-amino acids, β-amino acids and/or other unnatural amino acids (e.g. amino acids with unnatural side chains) are included in the term “peptide”. For the purposes of the present invention, the term “peptide” is intended to include peptidyl analogues comprising modified amino acids. Such modifications may comprise the introduction or presence of a substituent in a ring or chain; the introduction or presence of an “extra” functional group such as an amino, hydrazino, carboxyl, formyl (aldehyde) or keto group, or another moiety; and the absence or removal of a functional group or other moiety. The term also includes analogues modified in the amino- and/or carboxy termini, such as peptide amides and N-substituted amides, peptide hydrazides, N-substituted hydrazides, peptide esters, and their like, and peptides that do not comprise the amino-terminal —NH₂ group or that comprise e.g. a modified amino-terminal amino group or an imino or a hydrazino group instead of the amino-terminal amino group, and peptides that do not comprise the carboxy-terminal carboxyl group or comprise a modified group instead of it, and so on.

Some examples of possible reaction types that can be used to modify peptides, forming “peptidyl analogues”, are e.g., cycloaddition, condensation and nucleophilic addition reactions as well as esterification, amide formation, formation of substituted amides, N-alkylation, formation of hydrazides, salt formation. Salt formation may be the formation of any type of salt, such as alkali or other metal salt, ammonium salt, salts with organic bases, acid addition salts etc. Peptidyl analogues may be synthesized either from the corresponding peptides or directly (via other routes).

Compounds that are structural or functional analogues of the peptides of the invention may be compounds that do not consist of amino acids or not of amino acids alone, or some or all of whose building blocks are modified amino acids. Different types of building blocks can be used for this purpose, as is well appreciated by those skilled in the art. The function of these compounds in biological systems is essentially similar to the function of the peptides. The resemblance between these compounds and the original peptides is thus based on structural and functional similarities. Such compounds are called peptidomimetic analogues, as they mimic the function, conformation and/or structure of the original peptides and, for the purposes of the present invention, they are included in the term “peptide”.

A functional analog of a peptide according to the present invention is characterized by a binding ability with respect to the binding to tumors, tumor tissue, tumor cells or tumor endothelium which is essentially similar to that of the peptides they resemble.

For example, compounds like benzolactam or piperazine containing analogues based on the primary sequence of the original peptides can be used (Adams et al., 1999; Nakanishi and Kahn, 1996; Houghten et al., 1999; Nargund et al., 1998). A large variety of types of peptidomimetic substances have been reported in the scientific and patent literature and are well known to those skilled in the art. Peptidomimetic substances (analogues) may comprise for example one or more of the following structural components: reduced amides, hydroxyethylene and/or hydroxyethylamine isosteres, N-methyl amino acids, urea derivatives, thiourea derivatives, cyclic urea and/or thiourea derivatives, poly(ester imide)s, polyesters, esters, guanidine derivatives, cyclic guanidines, imidazoyl compounds, imidazolinyl compounds, imidazolidinyl compounds, lactams, lactones, aromatic rings, bicyclic systems, hydantoins and/or thiohydantoins as well as various other structures. Many types of compounds for the synthesis of peptidomimetic substances are available from a number of commercial sources (e.g. Peptide and Peptidomimetic Synthesis, Reagents for Drug Discovery, Fluka ChemieGmbH, Buchs, Switzerland, 2000 and Novabiochem 2000 Catalog, Calbiochem-Novabiochem AG, Läufelfingen, Switzerland, 2000). The resemblance between the peptidomimetic compounds and the original peptides is based on structural and/or functional similarities. Thus, the peptidomimetic compounds mimic the properties of the original peptides and, for the purpose of the present application, their binding ability is similar to the peptides that they resemble. Peptidomimetic compounds can be made up, for example, of unnatural amino acids (such as D-amino acids or amino acids comprising unnatural side chains, or of β-amino acids etc.), which do not appear in the original peptides, or they can be considered to consist of or can be made from other compounds or structural units. Examples of synthetic peptidomimetic compounds comprise N-alkylamino cyclic urea, thiourea, polyesters, poly(ester imide)s, bicyclic guanidines, hydantoins, thiohydantoins, and imidazol-pyridino-inoles (Houghten et al. 1999 and Nargund et al., 1998). Such peptidomimetic compounds can be characterized as being “structural or functional analogues” of the peptides of this invention.

For the purpose of the present invention, the term “targeting unit” stands for a compound, a peptide, capable of selectively targeting and selectively binding to tumors, and, preferably, also to tumor stroma, tumor parenchyma and/or extracellular matrix of tumors. Another term used in the art for this specific association is “homing”. Tumor targeting means that the targeting units specifically bind to tumors when administered to a human or animal body. More specifically, the targeting units may bind to a cell surface, to a specific molecule or structure on a cell surface or within the cells, or they may associate with the extracellular matrix present between the cells. The targeting units may also bind to the endothelial cells or the extracellular matrix of tumor vasculature. The targeting units may bind also to the tumor mass, tumor cells and extracellular matrix of metastases.

Generally, the terms “targeting” or “binding” stand for adhesion, attachment, affinity or binding of the targeting units of this invention to tumors, tumor cells and/or tumor tissue to the extent that the binding can be objectively measured and determined e.g., by peptide competition experiments in vivo or ex vivo, on tumor biopsies in vitro or by immunological stainings in situ, or by other methods known by those skilled in the art. The exact mechanism of the binding of targeting units according to the present invention is not known. Tageting peptides according to the present invention are considered to be “bound” to the tumor target in vitro, when the binding is strong enough to withstand normal sample treatment, such as washes and rinses with physiological saline or other physiologically acceptable salt or buffer solutions at physiological pH, or when bound to a tumor target in vivo long enough for the effector unit to exhibit its function on the target.

The binding of the present targeting agents or targeting units to tumors is “selective” meaning that they do not bind to normal cells and organs, or bind to such to a significantly lower degree as compared to tumor cells and organs.

Pharmaceutically and diagnostically acceptable salts of the targeting units and agents of the present invention include salts, esters, amides, hydrazides, N-substituted amides, N-substituted hydrazides, hydroxamic acid derivatives, decarboxylated and N-substituted derivatives thereof. Suitable pharmaceutically acceptable salts are readily acknowledged by those skilled in the art.

Targeting Motifs According to the Present Invention

It has now surprisingly been found that a three-amino-acid motif Dd-Ee-Ff, wherein Dd-Ee-Ff is either Aa-Bb-Cc or Cc-Bb-Aa, and

Aa is isoleucine, leucine or tert-leucine, or a structural or functional analogue thereof;

Bb is arginine, homoarginine or canavanine, or a structural or functional analogue thereof; and

Cc is serine or homoserine, or a structural or functional analogue thereof, targets and exhibits selective binding to tumors and cancers and tumor cells and cancer cells.

Aa according to the present invention may comprise in its sidechain a branched, non-branched or alicyclic structure with at least two siminal or different atoms selected from the group consisting of carbon, silicon, halogen bonded to carbon, ether-oxygens and thioether-sulphur. The analogue may be selected from the group consisting of branched, non-branched or cyclic non-aromatic, lipophilic and hydrophobic amino acids or amino acid analogues or derivatives or structural and/or functional analogues thereof; amino acids or carboxylic acids or amino acid analogues or derivatives or carboxylic acid analogues or derivatives having one or more lipophilic carborane-type or other lipophilic boron-containing side chains or other lipophilic cage-type structures.

Aa may be selected from the group consisting of:

1) α-amino acids whose side chain is one of the following:

ethyl
propyl
1-methylpropyl (the side chain of isoleucine)
2-methylpropyl (the side chain of leucine)
2,2-dimethylpropyl
1-ethylpropyl
tert-butyl
tert-pentyl
3-methylbutyl
2-methylbutyl
methylbutyl
ethylbutyl
2-ethylbutyl
cyclohexyl
2-methylcyclohexyl
cyclopentyl
2-methylcyclopentyl
3-methylcyclohexyl
cyclobutyl
cyclopropyl
2-methylcyclopropyl
methoxyethyl
methoxyethyl
methoxymethyl
ethoxymethyl
2-ethoxyethyl
1-ethoxyethyl
2-methoxypropyl
2,2-dimethoxypropyl
1-methylpropyl
1-methylbutyl
1-methylpentyl
1,1-dimethylpropyl
1,1-dimethylbutyl
1,1-dimethylpentyl
1,2-dimethylpropyl
1-cyclopropylethyl
2-cyclopropylethyl
cyclopropylmethyl
1-cyclopropylethyl
1-cyclopropylpropyl
2-cyclopropylpropyl
3-cyclopropylpropyl
any cyclobutylalkyl
1-ethylpropyl
1-methylethyl
other mono-, di-, tri- or oligoalkyl-alkyl
other cyclic alkyl or substituted cyclic alkyl or alkyl that is substituted with one or more substituted or unsubstituted cycloalkyl group(s) and optionally one or more alkyl group(s)
allyl
vinyl
1-methylallyl
1-ethylallyl
1-ethylvinyl
1-propenyl
1-methyl-1-propenyl
methyl-1-propenyl
methyl-1-propenyl
1-ethyl-1-propenyl
ethyl-1-propenyl
ethyl-1-propenyl
1-methyl-1-butenyl
methyl-1-butenyl
methyl-1-butenyl
1-ethyl-1-butenyl
2-ethyl-1-butenyl
ethyl-2-butenyl
ethyl-2-butenyl
ethyl-3-butenyl
ethyl-3-butenyl
ethyl-3-butenyl
   2) any of the following carboxylic acids, including any optical isomers thereof:
4-methylpentanoic acid
3-methylpentanoic acid
4,4-dimethylpentanoic acid
3,4-dimethylpentanoic acid
3,3-dimethylpentanoic acid
3-methylhexanoic acid
4-methylhexanoic acid
5-methylhexanoic acid
2-ethylpentanoic acid
3-ethylpentanoic acid
4-ethylpentanoic acid
2-cyclopropylpentanoic acid
3-cyclopropylpentanoic acid
4-cyclopropylpentanoic acid
2-methylbutanoic acid
3-methylbutanoic acid
4-methylbutanoic acid
2-cyclopropylbutanoic acid
3-cyclopropylbutanoic acid
4-cyclopropylbutanoic acid
   3) any optical and geometrical isomer of any of the following compounds:
2-amino-4-methyl-3-pentenoic acid
2-amino-4-methyl-4-pentenoic acid
2-amino-5-methyl-3-hexenoic acid
2-amino-5-methyl-4-hexenoic acid
2-amino-5-methyl-5-hexenoic acid and
   4) aminosubstituted (N-substituted) analogues of the amino-comprising compounds of points 1 and 3 that bear at the amino group
one methyl, ethyl, propyl, isopropyl or other alkyl group
one cycloalkyl group
one 9-fluorenylmethyloxycarbonyl (FMOC) group
one benzyloxycarbonyl (Cbz) group
one tert-butyloxycarbonyl (BOC) group
two identical, similar and/or different groups selected from the ones mentioned above in this point (point 4).

Aa may also be an α-amino acid (either L- or D-amino acid) of the formula R¹—CR²(NH₂)—COOH wherein the side chain R¹ is selected from the side chains listed above, and the side chains R² is selected from the group consisting of: hydrogen, methyl, ethyl, propyl.

Bb according to the present invention may be selected from the group consisting of amino acids or structural or functional analogues thereof containing one or more guanyl groups, aminodino groups or their analogues and derivatives and structural or functional equivalents; one or more groups containing at least two nitrogen atoms each and have or can gain a delocalized positive charge.

Bb may be selected from the group of compounds of the following formula: 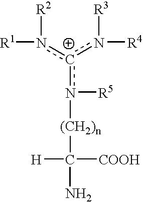
wherein R1-R5 is hydrogen or methyl, R2 and R3 may form —CH2-CH2- and n is 1-6.

Preferably, Bb is the L- or D-form of
arginine,
homoarginine,
canavanine,
2-amino-8-guanidino-octanoic acid,
2-amino-7-guanidino-octanoic acid,
2-amino-6-guanidino-octanoic acid,
2-amino-5-guanidino-octanoic acid,
2-amino-7-guanidino-heptanoic acid,
2-amino-6-guanidino-heptanoic acid,
2-amino-5-guanidino-heptanoic acid,
2-amino-4-guanidino-heptanoic acid,
2-amino-5-guanidino-hexanoic acid,
2-amino-4-guanidino-hexanoic acid,
2-amino-3-guanidino-hexanoic acid,
2-amino-4-guanidino-pentanoic acid,
2-amino-3-guanidino-pentanoic acid.

Cc according to the present invention may be selected from the group consisting of amino acids, amino alcohols, diamino alcohols, tri-, oligo- and polyamino alcohols and amino acid analogues, derivatives and structural or functional analogues thereof, comprising one or more hydroxyl group(s), esterified hydroxyl group(s), methoxyl group(s) and/or other etherified hydroxyl (ether) groups.

Cc as defined above may be serine or homoserine or a structural or functional analogue thereof, comprising at least one hydroxyl group; or may be selected from the group consisting of:

any other monoaminocarboxylic acid comprising at least one alcoholic hydroxyl group

any carboxylic acid comprising at least one alcoholic hydroxyl group

any other aminocarboxylic acid comprising an aliphatic or other side chain that comprises one or more alcoholic hydroxyl (OH) function(s) and/or esterified hydroxyl function(s).

Preferably, Cc is the L- or D-form of
serine,
homoserine,
2-amino-7-hydroxyheptanoic acid,
2-amino-5-hydroxypentanoic acid,
2-amino-6-hydroxyhexanoic acid,
2-amino-8-hydroxyoctanoic acid,
   or any other hydroxy-2-aminocarboxylic acid.

Alternatively, the motif Aa-Bb-Cc, as a whole, according to the present invention is a structural or functional analogue of a structure where Aa, Bb and Cc are as defined above.

Preferred embodiments of the present invention include tumor targeting motifs Aa-Bb-Cc selected from those given in Table 1 as well as structural and functional analogues thereof.

TABLE 1

| | Aa | Bb | Cc |
|---|---|---|---|
| 1 | L-isoleucine | L-arginine | L-serine |
| 2 | ″ | ″ | L-homoserine |
| 3 | D-isoleucine | D-arginine | D-serine |
| 4 | ″ | ″ | D-homoserine |
| 5 | L-leucine | L-arginine | L-serine |
| 6 | ″ | ″ | L-homoserine |
| 7 | D-leucine | D-arginine | D-serine |
| 8 | ″ | ″ | D-homoserine |
| 9 | L-isoleucine | L-homoarginine | L-serine |
| 10 | ″ | ″ | L-homoserine |
| 11 | D-isoleucine | D-homoarginine | D-serine |
| 12 | ″ | ″ | D-homoserine |
| 13 | L-leucine | L-homoarginine | L-serine |
| 14 | ″ | ″ | L-homoserine |
| 15 | D-leucine | D-homoarginine | D-serine |
| 16 | ″ | ″ | D-homoserine |
| 17 | L-2-aminopentanoic acid | L-arginine | L-serine |
| 18 | D-2-aminopentanoic acid | D-arginine | D-serine |
| 19 | L-2-aminopentanoic acid | L-arginine | L-homoserine |
| 20 | D-2-aminopentanoic acid | D-arginine | D-homoserine |
| 21 | L-2-aminohexanoic acid | L-arginine | L-serine |
| 22 | D-2-aminohexanoic acid | D-arginine | D-serine |
| 23 | L-2-aminohexanoic acid | L-arginine | L-homoserine |
| 24 | D-2-aminohexanoic acid | D-arginine | D-homoserine |
| 25 | L-2-aminoheptanoic acid | L-arginine | L-serine |
| 26 | D-2-aminoheptanoic acid | D-arginine | D-serine |
| 27 | L-2-aminoheptanoic acid | L-arginine | L-homoserine |
| 28 | D-2-aminoheptanoic acid | D-arginine | D-homoserine |
| 29 | L-2-amino-2-ethylbutanoic acid | L-arginine | L-serine |
| 30 | D-2-amino-2-ethylbutanoic acid | D-arginine | D-serine |
| 31 | L-2-amino-2-ethylbutanoic acid | L-arginine | L-homoserine |
| 32 | D-2-amino-2-ethylbutanoic acid | D-arginine | D-homoserine |
| 33 | L-isoleucine | L-arginine | 2-amino-7-hydroxyheptanoic acid |
| 34 | D-isoleucine | D-arginine | 2-amino-7-hydroxyheptanoic acid |
| 35 | L-leucine | D-arginine | 2-amino-7-hydroxyheptanoic acid |
| 36 | D-leucine | D-arginine | 2-amino-7-hydroxyheptanoic acid |
| 37 | L-isoleucine | L-arginine | L-2-amino-5-hydroxypentanoic acid |
| 38 | D-isoleucine | D-arginine | D-2-amino-5-hydroxypentanoic acid |
| 39 | L-leucine | L-arginine | L-2-amino-5-hydroxypentanoic acid |
| 40 | D-leucine | D-arginine | D-2-amino-5-hydroxypentanoic acid |
| 41 | L-isoleucine | L-arginine | L-2-amino-6-hydroxyhexanoic acid |
| 42 | D-isoleucine | D-arginine | D-2-amino-6-hydroxyhexanoic acid |
| 43 | L-leucine | L-arginine | L-2-amino-6-hydroxyhexanoic acid |
| 44 | D-leucine | D-arginine | D-2-amino-6-hydroxyhexanoic acid |
| 45 | L-2-aminopentanoic acid | L-homoarginine | L-serine |
| 46 | D-2-aminopentanoic acid | D-homoarginine | D-serine |
| 47 | L-2-aminopentanoic acid | L-homoarginine | L-homoserine |
| 48 | D-2-aminopentanoic acid | D-homoarginine | D-homoserine |
| 49 | L-2-aminohexanoic acid | L-homoarginine | L-serine |
| 50 | D-2-aminohexanoic acid | D-homoarginine | D-serine |
| 51 | L-2-aminohexanoic acid | L-homoarginine | L-homoserine |
| 52 | D-2-aminohexanoic acid | D-homoarginine | D-homoserine |
| 53 | L-2-aminoheptanoic acid | L-homoarginine | L-serine |
| 54 | D-2-aminoheptanoic acid | D-homoarginine | D-serine |
| 55 | L-2-aminoheptanoic acid | L-homoarginine | L-homoserine |
| 56 | D-2-aminoheptanoic acid | D-homoarginine | D-homoserine |
| 57 | L-2-amino-2-ethylbutanoic acid | L-homoarginine | L-serine |
| 58 | D-2-amino-2-ethylbutanoic acid | D-homoarginine | D-serine |
| 59 | L-2-amino-2-ethylbutanoic acid | L-homoarginine | L-homoserine |
| 60 | D-2-amino-2-ethylbutanoic acid | D-homoarginine | D-homoserine |
| 61 | L-isoleucine | L-homoarginine | 2-amino-7-hydroxyheptanoic acid |
| 62 | D-isoleucine | D-homoarginine | 2-amino-7-hydroxyheptanoic acid |
| 63 | L-leucine | D-homoarginine | 2-amino-7-hydroxyheptanoic acid |
| 64 | D-leucine | D-homoarginine | 2-amino-7-hydroxyheptanoic acid |
| 65 | L-isoleucine | L-homoarginine | L-2-amino-5-hydroxypentanoic acid |
| 66 | D-isoleucine | D-homoarginine | D-2-amino-5-hydroxypentanoic acid |
| 67 | L-leucine | L-homoarginine | L-2-amino-5-hydroxypentanoic acid |
| 68 | D-leucine | D-homoarginine | D-2-amino-5-hydroxypentanoic acid |
| 69 | L-isoleucine | L-homoarginine | L-2-amino-6-hydroxyhexanoic acid |
| 70 | D-isoleucine | D-homoarginine | D-2-amino-6-hydroxyhexanoic acid |
| 71 | L-leucine | L-homoarginine | L-2-amino-6-hydroxyhexanoic acid |
| 72 | D-leucine | D-homoarginine | D-2-amino-6-hydroxyhexanoic acid |

Thus, typical and preferred characteristics of Aa include lipofilicity, hydrophobicity and aliphatic character in at least one side chain, wheras Bb includes a delocalized positive charge and Cc has the ability of participating in OH-binding.

Especially preferred motifs Dd-Ee-Ff according to the present invention are leucine-arginine-serine (LRS) and serine-arginine-leucine (SRL).

The motifs Dd-Ee-Ff according to the present invention may form part of a larger structure, such as a peptide or some other structure. When the compound or structure in question comprises more than one motif Dd-Ee-Ff, the orientation and direction of the motifs may vary.

Targeting Units According to the Present Invention

It has also been found that peptides and structural or functional analogues thereof comprising a tumor targeting motif according to the present invention target to and exhibit selective binding to tumor cells and tissues. Peptides comprising a tumor targeting motif according to the present invention and, up to four additional amino acid residues or analogues thereof, likewise exhibit such targeting and selective binding and are especially preferred embodiments of the present invention.

Such peptides are highly advantageous for use as targeting units according to the present invention, e.g., because of their small size and their easy, reliable and cheap synthesis. Due to the small size of the peptides according to the present invention, the purification, analysis and quality control is easy and commercially useful.

Preferred tumor targeting units according to the present invention comprise a tumor targeting motif Dd-Ee-Ff as defined above, and additional residues selected from the group consisting of:

natural amino acids;

unnatural amino acids;

amino acid analogues comprising maximally 30 non-hydrogen atoms and an unlimited number of hydrogen atoms; and

other structural units and residues whose molecular weight and/or formula weight is maximally 270;

wherein

the number of said additional residues ranges from 0 to 4, preferably from 2 to 4, more preferably 2.

Cyclic peptides are usually more stable in vivo and in many other biological systems than are their non-cyclic counterparts, as is known in the art. It has now, however, surprisingly been found that the targeting property of the small peptides according to the present invention is more pronounced when the targeting unit is cyclic or contained in a cyclic structure.

Preferred targeting units according to the present invention may comprise a sequence
Cy-Rrₙ-Dd-Ee-Ff-Rrₘ-Cyy
wherein,
Dd-Ee-Ff is a tumor targeting motif Aa-Bb-Cc or Cc-Bb-Aa;
Rr is an amino acid residue or a structural or functional analogue thereof;
n and m are 0, 1 or 2, and the sum of n and m does not exceed two; and
Cy and Cyy are entities capable of forming a cyclic structure.

Preferred targeting units are such, where Rr is any amino acid residue, except histidine, lysine or tryptophane. Especially preferred are targeting units wherein Rr is R or G.

Preferred structures are such where Cy and Cyy are amino acids or analogues thereof containing a thiol group, such as homocysteine or cysteine or analogues thereof, or another structure with a molecular weight of no more than 270, comprising a thiol group or an oxidized thiol group. One preferred cyclic structure type is characterized by the presence of a disulphide bond (e.g., between cysteine moieties). Non-limiting examples of cyclic structures are, for example, compounds of the formula: 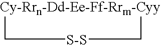
where Cy-S—S-Cyy indicates a cystine. Because of the easy availability and low price of cysteine, this type of structure is a preferred one.

The —S—S— bridge need not, however, be between cysteine units but may also exist between other amino acids or other moieties containing —SH groups. Such structures may comprise more than one Dd-Ee-Ff motif between the cysteine units, and may comprise additional amino acids and structural or functional analogues thereof outside the cyclic structure.

Highly preferred targeting units according to the present invention having a cyclic structure by virtue of a disulphide bridge, are CLRSC (SEQ ID NO. 1) and CSRLC (SEQ ID NO. 2).

Other preferred possibilities of forming the cyclic structure are the formation of an amide bond to give a lactam or an ester bond to give a lactone bond.

Preferred structures are thus compound of the general formula
Cy-Rrₙ-Dd-Ee-Ff-Rrₘ-Cyy
as defined above, and wherein Cy and Cyy are residues capable of forming a lactam bond, such as aspartic acid (D), glutamic acid (E), lysin (K), ornithine (O) or analogues thereof comprising no more than 12 carbon atoms.

Lactams can be of several subtypes, such as “head to tail” (carboxy terminus plus amino terminus), “head to side chain” and “side chain to head” (carboxy or amino terminus plus one side chain amino or carboxyl group) and “side chain to side chain” (amino groug of one side chain and carboxys group of another side chaine).

Highly preferred targeting units according to the present invention having a cyclic structure by virtue of a lactam bridge, are DLRSK (SEQ ID NO. 3), DLRSGRK (SEQ ID NO. 4) and DRGLRSK (SEQ ID NO. 5), OLRSE (SEQ ID NO. 6), KLRSD (SEQ ID NO. 7).

Targeting Agents According to the Present Invention

It has also been found that targeting agents comprising at least one tumor targeting unit according to the present invention, and at least one effector unit, target to and exhibit selective binding to cancer cells and tissues as well as endothelial cells.

The tumor targeting agents according to the present invention may optionally comprise unit(s) such as linkers, solubility modifiers, stabilizers, charge modifiers, spacers, lysis or reaction or reactivity modifiers, internalizing units or internalization enhancers or membrane interaction units or other 12 local route, attachment, binding and distribution affecting units. Such additional units of the tumor targeting agents according to the present invention may be coupled to each other by any means suitable for that purpose

Many possibilities are known to those skilled in the art for linking structures, molecules, groups etc. of the types in question or of related types, to each other. The various units may be linked either directly or with the aid of one or more identical, similar and/or different linker units. The tumor targeting agents of the invention may have different structures such as any of the non-limiting types schematically shown below: 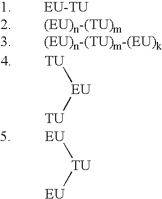
where EU indicates “effector unit” and TU indicates “targeting unit” and n, m and k are independently any integers except 0.

In the targeting agents according to the present invention, as in many other medicinal and other substances, it may be wise to include spacers or linkers, such as amino acids and their analogues, such as long-chain omega-amino acids, to prevent the targeting units from being ‘disturbed’, sterically, electronically or otherwise hindered or ‘hidden’ by effector units or other unit of the targeting agent.

In targeting agents according to the present invention it may be useful for increased activity to use dendrimeric or cyclic structures to provide a possiblility to incorporate multiple effector units or additional units per targeting unit.

Preferred targeting agents according to the present invention comprise a structure
Ef-TU-Eff, wherein
TU is a targeting unit according to the present invention as defined abover; and
Ef and Eff are selected from the group consisting of:
effector units, linker units, solubility modifier units, stabilizer units, charge modifier units, spacer units, lysis and/or reaction and/or reactivity modifier units, internalizing and/or internalization enhancer and/or membrane interaction units and/or other local route and/or local attachment/local binding and/or distribution affecting units, adsorption enhancer units, and other related units; and
peptide sequences and other structures comprising at least one such unit; and
peptide sequences comprising no more than 20, preferably no more than 12, more preferably no more than 6, natural and/or unnatural amino acids; and
natural and unnatural amino acids comprising no more than 25 non-hydrogen atoms and an unlimited number of hydrogen atoms;
as well as salts, esters, derivatives and analogues thereof.
Effector Units

For the purposes of this invention, the term “effector unit” means a molecule or radical or other chemical entity as well as large particles such as colloidal particles and their like; liposomes or microgranules. Suitable effector units may also consitute nanodevices or nanochips or their like; or a combination of any of these, and optionally chemical structures for the attachment of the constituents of the effector unit to each or to parts of the targeting agents. Effector units may also contain moieties that effect stabilization or solubility enhancement of the effector unit.

Preferred effects provided by the effector units according to the present invention are therapeutical (biological, chemical or physical) effects on the targeted tumor; properties that enable the detection or imaging of tumors or tumor cells for diagnostic purposes; or binding abilities that relate to the use of the targeting agents in different applications.

A preferred (biological) activity of the effector units according to the present invention is a therapeutic effect. Examples of such therapeutic activities are for example, cytotoxicity, cytostatic effect, ability to cause differentiation of cells or to increase their degree of differentiation or to cause phenotypic changes or metabolic changes, chemotactic activities, immunomodulating activities, pain relieving activities, radioactivity, ability to affect the cell cycle, ability to cause apoptosis, hormonal activities, enzymatic activities, ability to transfect cells, gene transferring activities, ability to mediate “knock-out” of one or more genes, ability to cause gene replacements or “knock-in”, antiangiogenic activities, ability to collect heat or other energy from external radiation or electric or magnetic fields, ability to affect transcription, translation or replication of the cell's genetic information or external related information; and to affect post-transcriptional and/or post-translational events.

Other preferred therapeutic approaches enabled by the effector units according to the present invention may be based on the use of thermal (slow) neutrons (to make suitable nuclei radioactive by neutron capture), or the administration of an enzyme capable of hydrolyzing for example an ester bond or other bonds or the administration of a targeted enzyme according to the present invention.

Examples of preferred functions of the effector units according to the present invention suitable for detection are radioactivity, paramagnetism, ferromagnetism, ferrimagnetism, or any type of magnetism, or ability to be detected by NMR spectroscopy, or ability to be detected by EPR (ESR) spectroscopy, or suitability for PET and/or SPECT imaging, or the presence of an immunogenic structure, or the presence of an antibody or antibody fragment or antibody-type structure, or the presence of a gold particle, or the presence of biotin or avidin or other protein, and/or luminescent and/or fluorescent and/or phosphorescent activity or the ability to enhance detection of tumors, tumor cells, endothelial cells and metastases in electron microscopy, light microscopy (UV and/or visible light), infrared microscopy, atomic force microscopy or tunneling microscopy, and so on.

Preferred binding abilities of an effector unit according to the present invention include, for example:
a) ability to bind to a substance or structure such as a histidine or other tag,
b) ability to bind to biotin or analogues thereof,
c) ability to bind to avidin or analogues thereof,
d) ability to bind to an enzyme or a modified enzyme,
e) ability to bind metal ion(s) e.g. by chelation,
f) ability to bind a cytotoxic, apoptotic or metobolism affectin substance or a substance capable of being converted in situ into such a substance,
g) ability to bind to integrins and other substances involved in cell adhesion, migration, or intracellular signaling,
h) ability to bind to phages,
i) ability to bind to lymphocytes or other blood cells,
j) ability to bind to any preselected material by virtue of the presence of antibodies or structures selected by biopanning,
k) ability to bind to material used for signal production or amplification,
l) ability to bind to therapeutic substance.

Such binding may be the result of e.g. chelation, formation of covalent bonds, antibody-antigen-type affinity, ion pair or ion associate formation, specific interactions of the avidin-biotin-type, or the result of any type or mode of binding or affinity.

One or more of the effector units or parts of them may also be a part of the targeting units themselves. Thus, the effector unit may for example be one or more atoms or nuclei of the targeting unit, such as radioactive atoms or atoms that can be made radioactive, or paramagnetic atoms or atoms that are easily detected by MRI or NMR spectroscopy (such as carbon-13). Further examples are, for example, boron-comprising structures such as carborane-type lipophilic side chains.

The effector units may be linked to the targeting units by any type of bond or structure or any combinations of them that are strong enough so that most, or preferably all or essentially all of the effector units of the targeting agents remain linked to the targeting units during the essential (necessary) targeting process, e.g. in a human or animal subject or in a biological sample under study or treatment.

The effector units or parts of them may remain linked to the targeting units, or they may be partly or completely hydrolyzed or otherwise disintegrated from the latter, either by a spontaneous chemical reaction or equilibrium or by a spontaneous enzymatic process or other biological process, or as a result of an intentional operation or procedure such as the administration of hydrolytic enzymes or other chemical substances. It is also possible that the enzymatic process or other reaction is caused or enhanced by the administration of a targeted substance such as an enzyme in accordance with the present invention.

One possibility is that the effector units or parts thereof are hydrolyzed from the targeting agent and/or hydrolyzed into smaller units by the effect of one or more of the various hydrolytic enzymes present in tumors (e.g., intracellularly, in the cell membrane or in the extracellular matrix) or in their near vicinity.

Taking into account that the targeting according to the present invention may be very rapid, even non-specific hydrolysis that occurs everywhere in the body may be acceptable and usable for hydrolysing one or more effector unit(s) intentionally, since such hydrolysis may in suitable cases (e.g., steric hindrance, or even without any such hindering effects) be so slow that the targeting agents are safely targeted in spite of the presence of hydrolytic enzymes of the body, as those skilled in the art very well understand. The formation of insoluble products and/or products rapidly absorbed into cells and/or bound to their surfaces after hydrolysis may also be beneficial for the targeted effector units and/or their fragments etc. to remain in the tumors or their closest vicinity.

In one preferred embodiment of the invention, the effector units may comprise structures, features, fragments, molecules or the like that make possible, cause directly or indirectly, an “amplification” of the therapeutic or other effect, of signal detection, of the binding of preselected substances, including biological material, molecules, ions, microbes or cells.

Such “amplification” may, for example, be based on one or more of the following non-limiting types:
the binding, by the effector units, of other materials that can further bind other substances (for example, antibodies, fluorescent antibodies, other “labelled” substances, substances such as avidin, preferably so that several molecules or “units” of the further materials can be bound per each effector unit;
the effector units comprise more than one entity capable of binding e.g. a protein, thus making direct amplification possible;
amplification in more than one steps.

Preferred effector units according to the present invention may be selected from the follwing group:
cytostatic or cytotoxic agents
apoptosis causing or enhancing agents
enzymes or enzyme inhibitors
antimetabolites
agents capable of disturbing membrane functions
radioactive or paramagnetic substances
substances comprising one or more metal ions
substances comprising boron, gadolinium, litium
substances suitable for neutron capture therapy
labelled substances
intercalators and substances comprising them
oxidants or reducing agents
nucleotides and their analogues
metal chelates or chelating agents.

In a highly preferred embodiment of the invention, the effector unit comprises alpha emittors.

In further preferred embodiments of the invention, the effector units may comprise copper chelates such as trans-bis(salicylaldoximaro) copper(II) and its analogues, or platinum compounds such cisplatin, carboplatin.

Different types of structures, substances and groups ar known that can be used to cause or enhance e.g., internalization into cells, including for example RQIKIWFQNRRMKWKK; Penetratin (Prochiantz, 1996), as well as stearyl derivatives (Promega Notes Magazine, 2000).

As an apoptosis-inducing structure, for example, the peptide sequence KLAKLAK that interacts with mitochondrial membranes inside cells, can be included Ellerby et al. (1999).

For use in embodiments of the present invention that include cell sorting and any related applications, the targeting units and agents of the invention can, for example, be used

a) coupled or connected to magnetic particles,

b) adsorbed, coupled, linked or connected to plastic, glass or other solid, porous, fibrous material-type or other surface(s) and the like,

c) adsorbed, covalently bonded or otherwise linked, coupled or connected into or onto one or more substance(s) or material(s) that can be used in columns and related systems

d) adsorbed, covalently bonded or otherwise linked, coupled or connected into or onto one or more substance(s) or material(s) that can be precipitated, centrifuged or otherwise separated or removed. Optional Units of the Targeting Agents According to the Present Invention

The targeting agents and targeting units of the present invention may optionally comprise further units, such as:

linker units coupling targeting units, effector units or other optional units of the present invention to each other;

solubility modifying units for modifying the solubility of the targeting agents or their hydrolysis product;

stabilizer units stabilizing the structure of the targeting units or agents during synthesis, modification, processing, storage or use in vivo or in vitro;

charge modifying units modifying the electrical charges of the targeting units or agents or their starting materials;

spacer units for increasing the distance between specific units of the targeting agents or their starting materials, to release or decrease steric hindrance or structural strain of the products;

reactivity modifyer units;

internalizing units or enhancer units for enhancing targeting and uptage of the targeting agents;

adsorption enhancer units, such as fat or water soluble structures enhancing absorption of the targeting agents in vivo; or other related units.

A large number of suitable linker units are known in the art. Examples of suitable linkers are:
1. for linking units comprising amino groups: cyclic anhydrides, dicarboxylic or multivalent, optinally activated or derivatized, carboxylic acids, compounds with two or more reactive halogens or compounds with at least one reactive halogen atom and at least one carboxyl group;
2. for linking units comprising carboxyl groups or derivatives thereof: compounds with at least two similar or different groups such as amino, substituted amino, hydroxyl, —NHNH₂ or substituted forms thereof, other known groups for the purpose (activators may be used);
3. for linking an amino group and a carboxyl group: for example amino acids and their activated or protected forms or derivatives;
4. for linking a formyl group or a keto group to another group are: a compound comprising e.g. at least one —N—NH₂ or —O—NH₂ or ═N—NH₂ or their like;
5. for linking several amino-comprising units: polycarboxylic substances such as EDTA, DTPA and polycarboxylic acids, anhydrides, esters and acyl halides;
6. for linking a substance comprising an amino group to a substance comprising either a formyl group or a carboxyl group: hydrazinocarboxylic acids or their like, preferably so that the hydrazino moiety or the carboxyl group is protected or activated, such as 4-(FMOC-hydrazino)benzoic acid;
7. for linking an organic structure to a metal ion: substances that can be coupled to the organic structure (e.g. by virtue of their COOH groups or their NH₂ groups) or that are integral parts of it, and that in addition comprise a polycarboxylic part for example an EDTA- or DTPA-like structure, peptides comprising several histidines or their like, peptides comprising several cysteines or other moieties comprising an —SH group each, and other chelating agents that comprise functional groups that can be used to link them to the organic structure.

A large variety of the above substances and other types of suitable linking agents are known in the art.

A large number of suitable solubility modifier units are known in the art. Suitable solubility modifier units comprise, for example:
for increasing aqeous solubility: molecules comprising SO₃⁻, O—SO₃⁻, COOH, COO⁻, NH₂, NH₃⁺, OH groups, guanidino or amidino groups or other ionic and ionizable groups and sugar-type structures;
for increasing fat solubility or solubility in organic solvents: units comprising (long) aliphatic branched or non-branched alkyl and alkenyl groups, cyclic non-aromatic groups such as the cyclohexyl group, aromatic rings and steroidal structures.

A large number of units known in the art can be used as stabilizer units, e.g. bulky structures (such as tert-butyl groups, naphthyl and adamantyl and related radicals etc.) for increasing steric hindrance, and D-amino acids and other unnatural amino acids (including β-amino acids, ω-amino acids, amino acids with very large side chains etc.) for preventing or hindering enzymatic hydrolysis.

Units comprising positive, negative or both types of charges can be used as charge modifier units, as can also structures that are converted or can be converted into units with positive, negative or both types of charges.

Spacer units may be very important, and the need to use such units depends on the other components of the structure (e.g. the type of biologically active agents used, and their mechanisms of action) and the synthetic procedures used.

Suitable spacer units may include for example long aliphatic chains or sugar-type structures (to avoid too high lipophilicity), or large rings. Suitable compounds are available in the art. One preferred group of spacer units are ω-amino acids with long chains. Such compounds can also be used (simultaneously) as linker units between an amino-comprising unit and a carboxyl-comprising unit. Many such compounds are commercially available, both as such and in the forms of various protected derivatives.

Units that are susceptible to hydrolysis (either spontaneous chemical hydrolysis or enzymatic hydrolysis by the body's own enzymes or enzymes administered to the patient) may be very advantageous in cases where it is desired that the effector units are liberated from the targeting agents e.g. for internalization, intra- or extracellularl DNA or receptor binding. Suitable units for this purpose include, for example, structures comprising one or more ester or acetal functionality, Various proteases may be used for the purposes mentioned. Many groups used for making pro-drugs may be suitable for the purpose of increasing or causing hydrolysis, lytic reactions or other decomposition processes.

The effector units, the targeting units and the optional units according to the present invention may simultaneously serve more than one function. Thus, for example, a targeting unit may simultaneously be an effector unit or comprise several effector units; a spacer unit may simultaneously be a linker unit or a charge modifier unit or both; a stabilizer unit may be an effector unit with properties different from those of another effector unit, and so on. An effector unit may, for example, have several similar or even completely different functions.

In one preferred embodiment of the invention, the tumor targeting agents comprise more than one different effector units. In that case, the effector units may be, for example, diagnostic and therapeutic units. Thus, for example, it is preferred to use, for boron neutron capture therapy, such agents whose effector units, in addition to comprising boron atoms, also can be detected or quantified in the patient in vivo after administration of the agent, in order to be able to ascertain that the agent has accumulated adequately in the tumor to be treated, or to optimize the timing of the neutron treatment, and so on. This goal may be achieved e.g. by using such a targeting agents according to the invention that comprise an effector unit comprising boron atoms (preferably isotope-enriched boron) and groups detectable e.g. by NMRI. Likewise, the presence of more than one type of therapeutically useful effector units may also be preferred. In addition, the targeting units and targeting agents may, if desired, be used in combination with one or more “classical” or other tumor therapeutic modalities such as surgery, chemotherapy, other targeting modalities, radiotherapy, immunotherapy etc.

Preparation of Targeting Units and Agents According to the Present Invention

The targeting units according to the present invention are preferably synthetic peptides. Peptides can be synthesized by a large variety of well-known techniques, such as solid-phase methods (FMOC-, BOC-, and other protection schemes, various resin types), solution methods (FMOC, BOC and other variants) and combinations of these. Even automated apparatuses/devices for the purpose are available commercially, as are also routine synthesis and purification services. All of these approaches are very well known to those skilled in the art. Some methods and materials are described, for example, in the following references:

Bachem AG, SASRIN™ (1999), The BACHEM Practise of SPPS (2000), Bachem 2001 catalogue (2001), Novabiochem 2000 Catalog (2000), Peptide and Peptidomimetic Synthesis (2000) and The Combinatorial Chemistry Catalog & Solid Phase Organic Chemistry (SPOC) Handbook 98/99. Peptide synthesis is exemplified also in the Examples.

As known in the art, it is often advisable, important and/or necessary to use one or more protecting groups, a large variety of which are known in the art, such as FMOC, BOC, and trityl groups and other protecting groups mentioned in the Examples. Protecting groups are often used for protecting amino, carboxyl, hydroxyl, guanyl and —SH groups, and for any reactive groups/functions.

As those skilled in the art well know, activation often involves carboxyl function activation and/or activation of amino groups.

Protection may also be orthogonal and/or semi/quasi/pseudo-orthogonal. Protecting and activating groups, substances and their uses are exemplified in the Examples and are described in the references cited herein, and are also described in a large number of books and other sources of information commonly known in the art (e.g. Protective Groups in Organic Synthesis, 1999).

Resins for solid-phase synthesis are also well known in the art, and are described in the Examples and in the above-cited references

Cyclic structures according to the present invention may be synthesized, for example, by methods based on the use of orthogonally protected amino acids. Thus, for example, one amino acid containing an orthogonally protected “extra” COOH function (e.g the (-allyl ester of N-(-FMOC-L-glutamic-acid, i.e., “FMOC-Glu-Oall”), or the (-tert-butyl ester of N-(-FMOC-L-glutamic acid (“FMOC-Glu-OtBu), or the (-4{N-[1-(4,4-dimethyl-2,6-dioxocyclohexylidene)-3-methylbutyl]-amino}benzyl ester of N-(-FMOC-L-glutamic acid (“FMOC-Glu-Odmab”) or the (-2-phenylisopropyl ester of N-(-FMOC-L-glutamic acid (“FMOC-Glu(O-2-PhiPr)-OH”), or related derivatives of other dicarboxylic amino acids, such as aspartic acid; or resin-bound forms of any of the aforementioned), and one amino acid with an orthogonally protected “extra” amino group (e.g N-(-FMOC-N-(4-methyltrityl-L-lysine (“FMOC-Lys(Mtt)-OH”) or the corresponding derivative of ornithine or some other diaminocarboxylic acid or a resin-bound form of one of these; resin-bound forms, however, not simultaneously with resin-bound forms of the orthogonally protected amino acids with “extra” COOH), may be incorporated in the structure and, after deprotection, the carboxyl and amino groups may be reacted, usually using activator(s). This type of methodology is well known and is described, for example, in the following references Novabiochem Catalog (2000), pp. 19-21 and 33 and specifically B9-B15, and in the references therein, Bachem 2001 catalogue (2001), pp. 31-32, Chan et al. (1995), Yue et al. (1993) and Hirschmann et al. (1998).

Suitable starting materials are available commercially, and further ones can be made by methods known in the art. D-amino acid derivatives can also be used in this methodology. Instead of “truly” orthogonal protective groups, also quasiorthogonal/semi-orthogonal/pseudoorthogonal protecting groups can be employed, as those skilled in the art understand.

Cyclic products made according to the above described methods are usually especially stable in biological milieu, and are thus preferred. This type of structures may be produced by any of the methods for the production of such structures (chemical, enzymatic or biological). Many such methods are well known for those skilled in the art. Cyclic structures of this type can be syntesized chemically with the aid of solid-phase synthesis but they can likewise be synthesized using solution methods or a combination of both, as those skilled in the art well know. Amino acids with an “extra” carboxyl or amino function suitable for cyclization purposes (when adequately protected) include (as non-limiting possibilities), for example, those with the structures shown below:

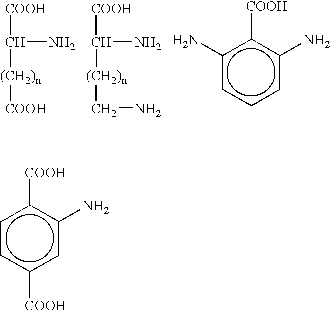

In solution cyclizations of any type, dilute solutions are normally advantageous, as is well known by those skilled in the art.

The targeting units and agents according to the present invention may also be prepared as fusion proteins or by other suitable recombinant DNA methods known in the art. Such an approach for preparing the peptides according to the present invention is preferred especially when the effector units and/or other optional units are peptides or proteins. One example of a useful protein effector unit is glutathion-S-transferase (GST).

Advantages of the Targeting Units and Targeting Agents of the Invention

There are acknowledged problems related to peptides intended for diagnostic or therapeutic use. One of these problems stem from the length of the sequence: the longer it grows, the more difficult the synthesis of the desired product becomes, especially if there are other synthetic problems such as the presence of difficult residues that require protection-deprotection and/or cause side reactions. The tendency to side-rections, and possible synthesis termination (that not only decreases the yield of the desired product if this is formed at all, but also gives rise to products with a wrong length of the peptide chain) and formation of serious amounts of harmful by-products. This is drastically increased if the desired sequence includes amino acids that require side-chain protection (e.g., basic side-chains such as those of lysine, histidine and tryptophan) and deprotection. These problems also make the purification of the desired peptides much more difficult and may make production of adequately purified material impossible.

As compared to known products that contain long and difficult-to-make sequences with problematic amino acid residues, the peptides of the present invention are clearly superior, as described in more detail below. Thus, the products and methods of the present invention and their use offer highly significant and very important advantages over the prior art.

The targeting units of this invention can be synthesized easily and reliably. An advantage as compared to many prior art peptides is that the targeting units and motifs of this invention do not comprise the problematic basic amino acids lysine and histidine, nor tryptophan, all of which may cause serious side-reactions in peptide synthesis, and, due to which the yield of the desired product might be lowered radically or even be impossible to obtain in adequate amounts or with adequate quality.

When present, histidine, lysine and tryptophan must be adequately protected using suitable protecting groups that remain intact during the synthesis prodecures. This may be very difficult and at least increases the costs and technical problems. Also costs are remarkably increased by the reagents and work-load and other costs of the deprotection steps and the costs per unit of desired product may be increased.

Because of their smaller size and thus drastically less steps in the synthesis, the peptides of the present invention are much easier and cheaper to produce than targeting peptides of the prior art.

As histidine is not needed in the products of the present invention, the risk of racemization of it is of no concern.

It is a great advantage not only for the economic synthesis of the products of the present invention but also for the purification and analysis and quality control that any racemization of histidine is outside consideration. It also makes any administration to humans and animals safer and more straightforward.

Because of their smaller size, the peptides of the present invention can also be purified much more reliably and easily and with much less labor and apparatus-time, and thus with clearly lower costs. Overall costs are thus drastically reduced and better products can be obtained and in greater amounts. Furthermore, the reliability of the purification is much better, giving less concern of toxic remainders and of fatal or otherwise serious side-effects in therapeutic and diagnostic applications.

Shorter synthesis protocols with relatively few steps produce less impurities, making the peptides of the present invention highly advantageous. The risks of toxic and even fatal impurities, allergens etc. are dramatically lowered and, in addition, purification is easier.

The analysis and thus the quality control of the products of the present invention is easier and less costly, than that of the longer and more ‘difficult’ peptide sequences. This increases the reliability of the analyses and of quality control.

As residues such as lysine are not present in the targeting unit, there is no the risk of the effector units being inadequately connected to such residues. This is a remarkable advantage.

The effector unit can easily be linked to the peptides and peptidyl analogues and peptidomimetic substances of the present invention using (outside the targeting motif for example protected lysine or ornithine as there is no risk of simultaneous reaction of any lysine residue in the targeting motif.

For cyclization of the peptides of the present invention, protected lysine or ornithine can be used, as the targeting motifs and units do not contain such amino acids. This is an enormous advantage.

In solid synthesis of targeting agents according to the present invention, the effector units and optional additional units may be linked to the targeting peptide when still connected to the resin without the risk that the removal of the protecting groups will cause destruction of additional unit. Similar advantage applies to solution syntheses.

Another important advantage of the present invention and its products, methods and uses according to it is the highly selective and potent targeting of the products.

As compared to targeted therapy using antibodies or antibody fragments, the products and methods of in the present invention are highly advantageous because of several reasons. Potential immunological and related risks are also obvious in the case of large biomolecules. Allergic reactions are of great concern with such products, in contrats to small synthetic molecules such as the targeting agents, units and motifs of the present invention.

As compared to targeting antibodies or antibody fragments, the products and methods described in the present invention are highly advantageous because their structure can be easily modified if needed or desired. Specific amino acids such as histidine, tryptophan, tyrosine and threonine can be omitted if dersired, and very few functional groups are necessary. On the other hand, it is possible, without disturbing the targeting effect, to include various different structural units, to specific desired properties that are of special value in specific applications

Use of Targeting Agents According to the Present Invention

The targeting units and targeting agents according to the present invention are useful in cancer diagnostics and therapy, as they selectively target to tumors in vivo, as shown in the examples. The effector unit may be chosen according to the desired effect, detection or therapy. The desired effect may also be achieved by including the effector in the targeting unit as such. For use in radiotherapy the targeting unit itself may be e.g., radioactively labelled.

The present invention also relates to diagnostic compositions comprising an effective amount of at least one targeting agent according to the present invention. In addition to the targeting agent, a diagnostic composition according to the present invention may, optionally, comprise carriers, solvents, vehicles, suspending agents, labelling agents and other additives commonly used in diagnostic compositions. Such diagnostic compositions are useful in diagnosing tumors, tumor cells and metastasis.

A diagnostic composition according to the present invention may be formulated as a liquid, gel or solid formulation, preferably as an aqueous liquid, containing a targeting agent according to the present invention in a concentration ranging from about 0.00001 μg/l to 25×10⁷ μg/l. The compositions may further comprise stabilizing agents, detergents, such as polysorbates and Tween, as well as other additives. The concentrations of these components may vary significantly depending on the formulation used. The diagnostic compositions may be used in vivo or in vitro.

The present invention also includes the use of the targeting agents and targeting units for the manufacture of pharmaceutical compositions for the treatment of cancer.

The present invention also relates to pharmaceutical compositions comprising a therapeutically effective amount of at least one targeting agent according to the present invention. The pharmaceutical compositions may be used to treat, prevent or ameliorate cancer diseases, by administering an therapeutically effective dose of the pharmaceutical composisiton comprising targeting agents or targeting units according to the present invention or therapeutically acceptable salts, esters or other derivatives thereof. The compositions may also include different combinations of targeting agents and targeting units together with labelling agents, imaging agents, drugs and other additives.

A therapeutically effective amount of a targeting agent according to the present invention may vary depending on the formulation of the pharmaceuticakl composition. Preferably, a composition according to the present invention may comprise a targeting agent in a concentration varying from about 0.00001 μg/l to 250 g/l, more preferably about 0.001 μg/l to 50 g/l, most preferably 0.01 μg/l to 20 g/l.

A pharmaceutical composition according to the present invention is useful for administration of a targeting agent according to the present invention. Pharmaceutical compositions suitable for peroral use, for intravenous or local injection, or infusion are particularly preferred. The pharmaceutical compositions may be used in vivo or ex vivo.

The preparations may be lyophilized and reconstituted before administration or may be stored for example as a solution, solutions, suspensions, suspension-solutions etc. ready for administration or in any form or shape in general, including powders, concentrates, frozen liquids, and any other types. They may also consist of separate entities to be mixed and, possibly, otherwise handled and/or treated etc. before use. Liquid formulations provide the advantage that they can be administered without reconstitution. The pH of the solution product is in the range of about 1 to about 12, preferably close to physiological pH. The osmolality of the solution can be adjusted to a preferred value using for example sodium chloride and/or sugars, polyols and/or amino acids and/or similar components. The compositions may further comprise pharmaceutically acceptable excipients and/or stabilizers, such as albumin, sugars and various polyols, as well as any acceptable additives, or other active ingredients such as chemotherapeutic agents.

The present invention also relates to methods for treating cancer, especially solid tumors by administering to a patient in need of such treatment a therapeutically efficient amount of a pharmaceutical composition according to the present invention.

Therapeutic doses may be determined empirically by testing the targeting agents and targeting units in available in vitro or in vivo test systems. Examples of such tests are given in the examples. Suitable therpeutically effective dosage may then be estimated from these experiments.

For oral administration it is important that the targeting units and targeting agent are stable and adequately absorbed from the intestinal tract.

The pharmaceutical compositions according to the present invention may be administered systemically, non-systemically, locally or topically, parenterally as well as non-parenterally, e.g. subcutaneously, intravenously, intramuscularly, perorally, intranasally, by pulmonary aerosol, by injection or infusion into a specific organ or region, buccally, intracranically or intraperitoneally.

Amounts and regimens for the administration of the tumor targeting agents according to the present invention can be determined readily by those with ordinary skill in the clinical art of treating cancer. Generally, the dosage will vary depending upon considerations such as: type of targeting agent employed; age; health; medical conditions being treated; kind of concurrent treatment, if any, frequency of treatment and the nature of the effect desired; gender; duration of the symptoms; and, counterindications, if any, and other variables to be adjusted by the individual physician. Preferred doses for administration to human patients targeting targeting units or agents according to the present invention may vary from about 0.000001 μg to about 40 mg per kg of body weight as a bolus or repeatedly, e.g., as daily doses.

The targeting units and targeting agents and pharmaceutical compositions of the present invention may also be used as targeting devices for delivery of DNA or RNA or structural and functional analogues thereof, such as phosphorothioates, or peptide nucleic acids (PNA) into tumors and their metastases or to isolated cells and organs in vitro; i.e. as tools for gene therapy both in vivo and in vitro. In such cases the targeting agents or targeting units may be parts of viral capsids or envelopes, of liposomes or other “containers” of DNA/RNA or related substances, or may be directly coupled to the DNA/RNA or other molecules mentioned above.

The present invention also includes kits and components for kits for diagnosing, detecting or analysing cancer or cancer cells in vivo and in vitro. Such kits comprise at least a targeting agent or targeting unit of this invention together with diagnostic entities enabling detection. The kit may comprise for example a targeting agent and/or a targeting unit coupled to a unit for detection by e.g. immunological methods, radiation or enzymatic methods or other methods known in the art.

Further, the targeting units and agents of this invention as well as the targeting motifs and sequences can be used as lead compounds to design peptidomimetics for any of the purposes described above.

Yet further, the targeting units and agents as well as the targeting motifs and sequences of the present invention, as such and/or as coupled to other materials, can be used for the isolation, purification and identification of the cells, molecules and related biological targets.

The following non-limiting examples illustrate the invention further.

### EXAMPLES

A list of reagents used in the examples below and reagent suppliers is included after the last numbered example.

### Example 1

Synthesis of a Targeting Unit (Peptide) LRS

A functionally protected, resin bound targeting unit (protected peptide), comprising targeting motif LRS, was synthesized using the method described in Example 2.

The following reagents were employed as starting materials (in this order):

Fmoc-Ser(tBu) Resin, Applied Biosystems Cat. No. 401429, 0.64 mmol/g

Fmoc-L-Arg(Pbf)-OH, CAS No. 154445-77-9, Applied Biosystems Cat. No. GEN911097, Molecular Weight: 648.8 g/mol

Fmoc-L-Leu-OH, CAS No. 35661-60-0, Applied Biosystems Cat. No. GEN911048, Molecular Weight: 353.4 g/mol

After the last cycle of the coupling process, a small sample of the resin bound peptide was subjected to Fmoc removal (steps 1-10 in Example 2), after which the peptide was cleaved off the resin by a three hours' treatment with the cleavage mixture and isolated as described in Example 2. The product (LRS) was identified with the aid of its positive mode MALDI-TOF mass spectrum, in which the M+1 ion of LRS was clearly predominant.

MALDI-TOF Data (LRS):

calculated molecular mass=374.44

Observed Signals:

375.30 M+H

397.22 M+Na

### Example 2

General Description of the Manual Solid Phase Peptide Synthesis and Mass Spectral Measurements Used for Synthesising Peptides Described in the Examples

All synthetic procedures were carried out in a sealable glass funnel equipped with a sintered glass filter disc of porosity grade between 2 and 4, a polypropene or phenolic plastic screw cap on top (for sealing), and two PTFE key stopcocks: one beneath the filter disc (for draining) and one at sloping angle on the shoulder of the screw-capped neck (for argon gas inlet).

The funnel was loaded with the appropriate solid phase synthesis resin and solutions for each treatment, shaken powerfully with the aid of a “wrist movement” bottle shaker (Gallenkamp™) for an appropriate period of time, followed by filtration effected with a moderate argon gas pressure.

The general procedure of one cycle of synthesis (=the addition of one amino acid unit) was as follows:

An appropriate Wang or Rink (Rink amide) resin, loaded with approximately 1 mmol of Fmoc-peptide (=peptide whose amino-terminal amino group was protected with the 9-fluorenylmethyloxycarbonyl group) consisting of two or more amino acid units, or with approximately 1 mmol of the appropriate Fmoc-amino acid (i.e., amino acid carrying the aforementioned protecting group; approximately 2 g of resin, 0.5 mmol/g) was treated in the way described below, each treatment step comprising shaking for 2.5 minutes with 30 ml of the solution or solvent indicated and filtration if not mentioned otherwise.

‘DCM’ means shaking with dichloromethane, and ‘DMF’ means shaking with N,N-dimethylformamide (DMF may be replaced by NMP, i.e. N-methylpyrrolidinone).

The steps of the treatment were:
1. DCM, shaking for 10-20 min
2. DMF
3. 20% (by volume) piperidine in DMF for 5 min
4. 20% (by volume) piperidine in DMF for 10 min
5. to 7. DMF
8. to 10. DCM
11. DMF
12. DMF solution of 3 mmol of activated amino acid (preparation described below), shaking for 2 hours
13. to 15. DMF
16 to 18. DCM

After the last treatment (18) argon gas was led through the resin for approximately 15 min and the resin was stored under argon (in the sealed reaction funnel if the synthesis was to continue with further units).

Activation of the 9-fluorenylmethyloxycarbonyl-N-protected amino acid (Fmoc-amino acid) to be added to the amino acid or peptide chain on the resin was carried out, using the reagents listed below, in a separate vessel prior to treatment step no. 12. Thus, the Fmoc-amino acid (3 mmol) was dissolved in approximately 10 ml of DMF, treated for 1 min with a solution of 3 mmol of HBTU dissolved in 6 ml of a 0.5 M solution of HOBt in DMF, and then immediately treated with 3 ml of a 2.0 M DIPEA solution for 5 min.

The activation reagents used for activation of the Fmoc-amino acid were as follows:

HBTU=2-(1H-benzotriazol-1-yl)-1,1,3,3-tetramethyluronium hexafluorophosphate, CAS No. [94790-37-1], Applied Biosystems Cat. No. 401091, molecular weight: 379.3 g/mol

HOBt=1-Hydroxybenzotriazole, 0.5 M solution in DMF, Applied Biosystems Cat. No. 400934

DIPEA=N,N-Diisopropylethylamine, 2.0 M solution in N-methylpyrrolidone, Applied Biosystems Cat. No. 401517

The procedure described above was repeated in several cycles using the appropriate different Fmoc-amino acids, carrying suitable protecting group(s), to produce a resin-bound source of the appropriate peptide (i.e., a “resin-bound” peptide). The procedure provides also a practical way of connecting certain effector and/or spacer and/or linker units and so on, for instance biotin or the Fmoc-Ahx (=6-(Fmoc-amino)-hexanoyl) moiety, to the resin-bound peptide.

Cleavage from the resin was carried out using the following reagent mixture:

trifluoroacetic acid (TFA) 92.5 vol-%

water 5.0 vol-%

ethanedithiol 2.5 vol-%.

After the removal of the protecting Fmoc group via steps 1. to 10. (as described in the general procedure above), the resin was treated with three portions of the above reagent mixture (each about 15 ml for 1 g of the resin), each for one hour. The treatments were carried out under argon atmosphere in the way described above. The TFA solutions obtained by filtration were then concentrated under reduced pressure using a rotary evaporator and were recharged with argon. Some diethyl ether was added and the concentration repeated. The concentrated residue was allowed to precipitate overnight under argon in dietyl ether in a refrigerator. The supernatant ether was removed and the precipitate rinsed with diethyl ether. For mass spectrum (MALDI-TOF+) determination, a sample of the precipitate was dissolved in solvents adequate for the spectral method, followed by filtration and, as needeed, dilution of the filtered solution. Further purification was done using reversed phase high-performance liquid chromatographic (HPLC) methods by means of a “Waters 600” pump apparatus using a C-18 type column of particle size 10 micrometers and a linear eluent gradient whose composition was changed during 30 minutes from 99.9% water/0.1% TFA to 99.9% acetonitrile/0.1% TFA. The dimensions of the HPLC columns were 25 cm×21.2 mm (Supelco cat. no. 567212-U) and 15 cm×10 mm (Supelco cat. no. 567208-U). Detection was based on absorbance at 218 nm and was carried out using a “Waters 2487” instrument.

The cleavage mixture described above also simultaneously removed the following protecting groups: trityl (Trt) as used for cysteine —SH protection; 2,2,4,6,7-pentamethyldihydrobenzofuran-5-sulfonyl (Pbf) as used for protection of side chain of arginine; the tert-butyl group (as an ester group on the carboxyl function; OtBu) as used for protection of the side-chain carboxyl group of glutamic acid and/or aspartic acid, and can normally be used also for removal of these protecting groups on analogous structures (thiol, guanyl, carboxyl). It did not cause Fmoc removal.

The cleavage procedure described above can be carried out also without the removal of the Fmoc group, to produce the amino terminal N-Fmoc-derivative of the peptide, or for a peptide linked to an effector unit (comprising no Fmoc).

Mass spectral method employed: Matrix Assisted Laser Desorption Ionization-Time of Flight (MALDI-TOF)

Type of the intrument: Bruker Biflex MALDI TOF mass spectrometer

Supplier of the instrument: Bruker Daltonik GmbH, Fahrenheit-strasse 4, D-28359 Bremen, Germany

MALDI-TOF positive ion reflector mode: External standards: Angiotensin 11 and ACTH(18-39)

Matrix: alpha-cyano-4-hydroxycinnamic acid (saturated solution in aqueous 50% acetonitrile containing 0.1% of trifluoroacetic acid).

The sample, together with the matrix, was dried onto the target plate under a gentle stream of warm air.

MALDI-TOF negative ion reflector mode: External standards: cholecystokinin and glucagon

Matrix: 2,4,6-trihydroxyacetophenone (3 mg/ml in 10 mM ammonium citrate in 50% acetonitrile).

The sample, mixed with the matrix, was immediately dried onto the target plate under vacuum.

Sample preparation: The specimen was mixed at a 10-100 pico-mol/microliter concentration with the matrix solution as described.

“Shooting” by nitrogen laser at wawelength 337 nm. The voltage of the probe plate was 19 kV in the positive ion reflector mode and −19 kV in the negative ion reflector mode.

General remarks about the spectra (concerning positive ion mode only): In all cases the M+1 (i.e. the one proton adduct M+H+) signal with its typical fine structure based on isotope satellites was clearly predominant. In almost all cases, the M+1 signal pattern was accompanied by a similar but markedly weaker band of peaks at M+23 (Na⁺ adduct). In addition to the bands at M+1 and M+23, also bands at M+39 (K⁺ adduct) or M+56 (Fe⁺ adduct) could be observed in many cases.

In case of substances with a low molecular mass, the ‘matrix signals’ (signals due to the constituents of the matrix/‘the ionization environment’) have been omitted (i.e., signals at 294 and 380 Da have been omitted).

The calculated molecular mass values reported within synthesis examples correspond to the most abundant isotopes of each element, i.e., the ‘exact masses’. The interpretations given for signals are only tentative.

### Example 3

General Procedures for I₂-Promoted Cyclization of Cystein Comprising Peptides Described in the Examples

The resin (1 g) was swelled on CH₂Cl₂ (15 ml) and stirred for 20 minutes. The solvent was removed by filtration and the resin was treated once with DMF (15 ml) for three minutes. After filtration, the resin-bound peptide (or targeting agent) was treated with iodine (5 molar equivalents) in DMF (10 ml) for 1 hour.

The DMF-iodine solution was removed by filtration and the residue was washed three times with DMF (15 ml) and three times with CH₂Cl₂ (15 ml) for 3 minutes each time.

In case that a ‘plain’ peptide (without the Fmoc group) was to be prepared, the Fmoc group was removed and the peptide was released from the resin according to the general procedure described in Example 2 and purified by reversed phase HPLC. In the case of targeting agents comprising no Fmoc group, the product was released from the resin and purified analogously.

Material used:

Iodine, CAS No. 7553-56-2, molecular weight: 253.81, Merck Art. No. 4760

### Example 4

Synthesis of Targeting Unit (Peptide) DLRSK

A functionally protected, resin bound targeting unit (protected peptide), comprising targeting motif LRS, was synthesized by means of manual synthesis as described in Example 2 above.

The following reagents were employed as starting materials (in this order):

Fmoc-Lys(Mtt)-resin, 0.68 mmol/g, Bachem Cat. No. D-2565.0005

Fmoc-L-Ser(tBu)-OH, CAS No. 71989-33-8, Perseptive Biosystems Cat. No.

GEN911062, Molecular Weight: 383.4 g/mol

Fmoc-L-Arg(Pbf)-OH

Fmoc-L-Leu-OH

Fmoc-Asp(2-phenylisopropyl ester)-OH, Molecular weight: 473.53 g/mol, Bachem Cat. No. B-2475.0005

After the last cycle of the coupling process, the still resin-bound targeting unit was subjected to the cyclization process in which an extra amide bond is formed as described in Example 21. After the cyclization process (macrolactam formation) a small sample of the resin (containing the still fully protected cyclized peptide) was subjected to the treatment described in Example 2 for Fmoc removal (steps 1-10 in that Example), after which the sample of peptide was cleaved from the resin by three hours' treatment with the cleavage mixture described in Example 2, and isolated as described in the same example.

Then, the product (DLRSK macrolactam) was identified with the aid of its positive mode MALDI-TOF mass spectrum, in which the M+1 ion of cyclic DLRSK was clearly predominant.

MALDI-TOF Data (Cyclic DLRSK):

calculated molecular mass=599.34

Observed Signals:

600.42 M+H

622.40 M+Na

638.29 M+K

Fmoc-DLRSK Macrolactam

Cyclic Fmoc-DLRSK was prepared and identified in analogous manner to cyclic DLRSK with the exeption of the final Fmoc removal that was omitted in this case.

MALDI-TOF Data (Cyclic Fmoc-DLRSK):

calculated molecular mass=821.41

Observed Signals:

822.60 M+H

844.62 M+Na

### Example 5

Synthesis of Targeting Unit (Peptide) DLRSGRK

The functionally protected, resin bound targeting unit (protected peptide), comprising targeting motif LRS, was synthesized by means of manual synthesis as described in Example 2 above.

The following reagents were employed as starting materials (in this order):

Fmoc-Lys(Mtt)-resin

Fmoc-L-Arg(Pbf)-OH

Fmoc-Gly-OH, CAS No. 29022-11-5, Novabiochem Cat. No. 04-12-1001, Molecular Weight: 297.3 g/mol

Fmoc-L-Ser(tBu)-OH

Fmoc-L-Arg(Pbf)-OH

Fmoc-L-Leu-OH

Fmoc-Asp(2-phenylisopropyl ester)-OH

After the last cycle of the coupling process, the still resin-bound targeting unit was subjected to the cyclization process in which an extra amide bond is formed as described in Example 21. After the cyclization process (macrolactam formation) a small sample of the resin (containing the still fully protected cyclized peptide and being suitable starting material for further synthesis e.g. biotinylation) was subjected to the treatment described in Example 2 for Fmoc removal (steps 1-10 in that Example), after which the sample of peptide was cleaved from the resin by three hours' treatment with the cleavage mixture described in Example 2, and isolated as described in the same Example.

Then, the product (cyclic DLRSGRK) was identified with the aid of its positive mode MALDI-TOF mass spectrum, in which the M+1 ion of cyclic DLRSK was clearly predominant.

MALDI-TOF Data (Cyclic DLRSGRK):

calculated molecular mass=812.46

Observed Signal:

813.69 M+H

### Example 6

Synthesis of Targeting Unit (Peptide) DRGLRSK (Cyclic by Virtue of Lactam Bridge)

The functionally protected, resin bound targeting unit (protected peptide), comprising targeting motif LRS, was synthesized by means of manual synthesis as described in Example 2 above.

The following reagents were employed as starting materials (in this order):

Fmoc-Lys(Mtt) Resin

Fmoc-L-Ser(tBu)-OH

Fmoc-L-Arg(Pbf)-OH

Fmoc-L-Leu-OH

Fmoc-Gly-OH

Fmoc-L-Arg(Pbf)-OH

Fmoc-Asp(2-phenylisopropyl ester)-OH

After the last cycle of the coupling process, the still resin-bound targeting unit was subjected to the cyclization process in which an extra amide bond is formed as described in Example 21. After the cyclization process (macrolactam formation) a small sample of the resin (containing the still fully protected cyclized peptide) was subjected to the treatment described in Example 2 for Fmoc removal (steps 1-10 in that Example), after which the sample of peptide was cleaved from the resin by three hours' treatment with the cleavage mixture described in Example 2, and isolated as described in the same Example.

Then, the product (DRGLRSK macrolactam) was identified with the aid of its positive mode MALDI-TOF mass spectrum, in which the M+1 ion of cyclic DRGLRSK was clearly predominant.

MALDI-TOF Data (Cyclic DRGLRSK):

calculated molecular mass=812.46

Observed Signal:

813.34 M+H

### Example 7

Synthesis of Targeting Unit (Peptide) AhxDLRSK, That is Cyclic by Virtue of Lactam Bridge

The functionally protected, resin bound targeting unit (protected peptide), comprising targeting motif LRS, was synthesized by means of manual synthesis as described in Example 2 above.

The following reagents were employed as starting materials (in this order):

Fmoc-Lys(Mtt)-resin

Fmoc-L-Ser(tBu)-OH

Fmoc-L-Arg(Pbf)-OH

Fmoc-L-Leu-OH

Fmoc-Asp(2-phenylisopropyl ester)-OH

Fmoc-6-aminohexanoic acid, (Fmoc-6-Ahx-OH), CAS No. 88574-06-5, Novabiochem Cat. No. 04-12-1111 A22837, Molecular Weight: 353.4 g/mol

After the last cycle of the coupling process, the still resin-bound targeting unit was subjected to the cyclization process in which an extra amide bond is formed as described in Example 21. After the cyclization process (macrolactam formation) a small sample of the resin (containing the still fully protected cyclized peptide) was subjected to three hours' treatment with the cleavage mixture described in Example 2. By that way a sample of peptide was cleaved from the resin and the protecting groups of side chains of that sample were removed with the exception of the final Fmoc removal that was omitted in this case. The sample was isolated as described in the same Example. Then, the product (DLRSK macrolactam) was identified with the aid of its positive mode MALDI-TOF mass spectrum, in which the M+1 ion of cyclic DLRSK was clearly predominant.

MALDI-TOF Data (Cyclic Fmoc-AhxDLRSK):

calculated molecular mass=938.50

Observed Signal:

939.50 M+1

### Example 8

Synthesis of Targeting Agent Fmoc2Dap-DLRSK (Dap=Diaminopropionyl), Comprising the Effector Unit Diaminopropionic Acid Coupled (Linked Directly, Without Specific Linker Units) via its Carboxyl Group to the N-Terminal Amino Group of the Peptide DLRSK by Virtue of an Amide Bond, and also Comprising the Targeting Unit DLRSK, that is Cyclic by Virtue of Lactam Bridge

The targeting agent was synthesized using manual synthesis as described in Example 2 above (analogously to the synthesis in Example 4 above, including cyclization). Next, the sequence DLRSK was continued with DL-2,3-Bis(Fmoc-amino)propionic acid by means of the general coupling methods described in Example 2.

The Preparation of DL-2,3-Bis(Fmoc-amino)propionic acid:

DL-2,3-diaminopropionic acid monohydrochloride was dissolved in 15 mL of aqueous 10% Na2CO3 solution. Then 7 mL of dioxane was added and the reaction mixture cooled to +4° C. Fmoc-chloride in 20 mL of dioxane was added and the reaction mixture stirred for one hour at +4° C. After continued stirring at room temperature overnight the reaction mixture was extracted with ethyl acetate that was then evaporated. The residue was triturated with n-hexane and washed with small amount of hot ethyl acetate to afford white solid that was dried in vacuo overnight.

Reagents Used:

DL-2,3-diaminopropionic acid monohydrochloride, CAS No. 54897-59-5, C3H8N2O2.HCl, Acros Organics, New Jersey USA; Ceel Belgium, Cat. No. 204670050

Fmoc-chloride; 9-fluorenylmethyl chloroformate 98%; C.A.S. no: 28920-43-6 Acros, cat no.: 170940250

MALDI-TOF Data (Fmoc2Dap-DLRSK, Cyclic):

calculated molecular mass=1129.53

Observed Signal:

1130.32 M+H

### Example 9

Synthesis of Targeting Unit (Peptide) (Fmoc-LRS)2Dapa. the Use of a Peptide Synthesis Resin with no Amino Acid Residue Pre-Coupled to it, and Derivatization of such a Resin with a Protected Amino Acid Derivative (Residue)

The synthesis of the targeting unit (peptide) (Fmoc-LRS)2Dapa [2,3-bis-(Fmoc-leucyl-arginyl-serinyl-amino)propionic acid] was performed by using the manual solid-phase peptide synthesis technique that is described in detail in Example 2.

The coupling (binding) of the first amino acid unit (residue) to the hydroxyl groups of a peptide synthesis resin (HMP type; for details, see the listing of materials given below) was carried out by means of the dichlorobenzoyl chloride method as applied to a derivative of 2,3-bis-aminopropionic acid whose amino functions were protected by the 9-fluorenylmethyloxycarbonyl (=Fmoc) group (the method of protection is described within Example 8). The following protocol was used:

The “empty” resin (resin with no amino acid residue; see below for producer and product number of the commercial resin) was first washed in the shaker described above (in Example 2) with N,N-dimethylformamide (DMF; 15 ml of DMF per 1 g of resin) for 20 min and was drained. After addition of five molecular equivalents (relative to the loading capacity of the resin) of the protected di-2,3-aminopropionic acid in DMF, after which 8 equivalents of pyridine were added, followed by shaking for about 3 minutes, without draining. Then, five equivalents of 2,6-dichlorobenzoyl chloride were added, and the mixture was shaken for 18 h at ambient temperature.

After the aforementioned treatment, the resin was drained and washed three times with DMF and dichloromethane as described in the general protocol in Example 2, followed by drying in an argon gas flow. The reagents used this far in the Example were:

HMP Resin, loading capacity: 1.16 mmol/g (as reported by the producer of the commercial product), Applied Biosystems Cat. No. 400957. Pyridine, Merck Art. No. 9728.

2,3-bis-(Fmoc-amino)propionic acid, preparation described in Example 8.

From this point on, the synthesis proceeded according to the general method decribed in Example 2 using reagent amounts relative to two molecular equivalents. The reagents used in this synthesis, not mentioned above or in Example 2 below, were:

Fmoc-L-Arg(Pbf)-OH

Fmoc-L-Leu-OH

The product, (Fmoc-LRS)2Dapa, after its isolation according to the general methods described in Example 2, was identified employing MALDI-TOF mass spectral analysis (positive ion mode) as described in detail in the general protocol in Example 2.

MALDI-TOF Data [(Fmoc-LRS)2Dapa]

calculated molecular mass=1260.63

Observed Signal:

1261.40 M+H

### Example 10

Synthesis of Targeting Agent Aoa-DLRSK (Aoa=Aminooxyacetyl=NH2OCH2CO), Comprising the Effector Unit Amino-Oxyacetic Acid Coupled (Linked Directly, Without Specific Linker Units) via its Carboxyl Group to the N-Terminal Amino Group of the Peptide DLRSK by Virtue of an Amide Bond, and also Comprising the Targeting Unit DLRSK, that is Cyclic by Virtue Lactam Bridge

The targeting agent was synthesized using manual synthesis as described in Example 2 above (analogously to the synthesis in Example 4 above, including cyclization). Next, the sequence DLRSK was continued with amino-oxyacetic acid by means of the general coupling methods described in Example 2.

Reagent Used:

Boc-amino-oxyacetic acid; Boc-NH—OCH2—COOH, Molecular weight: 191.2 g/mol, CAS No., Novabiochem Cat. No. 01-63-0060

MALDI-TOF Data (Aoa-DLRSK, Cyclic):

calculated molecular mass=674.37

Observed Signals:

673.54 M+H

### Example 11

Synthesis of Targeting Agent Bio-LRS (Bio=D-Biotin=Vitamin H), Comprising the Effector Unit D-Biotin Coupled (Linked Directly, Without Specific Linker Units) via its Carboxyl Group to the N-Terminal Amino Group of the Peptide LRS by Virtue of an Amide Bond, and also Comprising the Targeting Unit LRS

The targeting agent was synthesized using manual synthesis as described in Example 2 above (analogously to the synthesis in Example 1 above) and using the biotinylation procedure described in Example 13 below as the final coupling step. In this final coupling process, D-biotin was employed instead of a protected amino acid. D-biotin was not protected but was employed as such. The product was isolated and purified in the manner indicated in Example 2 and identified by positive-mode MALDI-TOF spectroscopy (M+1 ion clearly predominant).

MALDI-TOF Data (Bio-LRS):

calculated molecular mass=600.31

Observed Signals:

601.34 M+H

623.23 M+Na

639.25 M+K

### Example 12

Synthesis of Targeting Agent Bio-DLRSK (Bio=D-Biotin=Vitamin H), Comprising the Effector Unit D-Biotin Coupled (Linked Directly, Without Specific Linker Units) via its Carboxyl Group to the N-Terminal Amino Group of the Peptide DLRSK by Virtue of an Amide Bond, and also Comprising the Targeting Unit DLRSK, that is Cyclic by Virtue of an Amide Bond Between the Side Chains of the Outermost Members of the Sequence

The targeting agent was synthesized using manual synthesis as described in Example 2 above (analogously to the synthesis in Example 4 above, including cyclization) and using the biotinylation procedure described in Example 13 below as the final coupling step. In this final coupling process, D-biotin was employed instead of a protected amino acid. D-biotin was not protected but was employed as such. The product was isolated and purified in the manner indicated in Example 2 and identified by positive-mode MALDI-TOF spectroscopy (M+1 ion clearly predominant).

MALDI-TOF Data (Bio-DLRSK Cyclic):

calculated molecular mass=825.42

Observed Signals:

826.49 M+H

848.35 M+Na

### Example 13

General Procedure Employed in the Syntheses of Biotinylated Compounds [Targeting Agents Comprising One D-Biotin (Vitamin H) as an Effector Unit]

The appropriate protected peptide was synthesized on using solid-phase synthesis according to the general procedure described in Example 2. The peptide was not deprotected and also not removed from the resin. The resin-bound peptide was added to the reaction flask. The resin was swelled using CH2Cl2 (15 ml) and stirred for 20 minutes. The solvent was removed by filtration and the resin was treated once with DMF for three minutes. The peptide was deprotected using 20% piperidine solution in DMF (20 ml) and shaking therewith for 5, and the process was repeated using (now shaking for 10 minutes). The resin was washed three times with DMF (15 ml) and three times with CH2Cl2 (15 ml) and once with DMF (15 ml) for three minutes each time.

D-biotin (3 molar equivalents) in DMF (10 ml) (heterogenous suspension) was treated in a separate vessel with a 0.5 M solution of HBTU/HOBT in DMF (3 molar eq.) for one minute. Into the vessel was added a 2 M solution of di-isopropylethylamine in NMP (6 molar eq.). After the addition, the reaction mixture became homogenous. The mixture was added to the reaction apparatus and the apparatus was shaken for 2 hours.

The reaction mixture was then filtered and the residue was washed three times with DMF (15 ml) and three times with CH2Cl2 (15 ml) for 3 minutes each time.

In case that the peptide was to be both biotinylated as described herein and cyclized by an iodine treatment as described in Example 3, the cyclization was performed after the biotinylation procedure.

Material Used:

D-Biotin (Vitamin H), CAS No. 58-85-5, molecular weight: 244.3, Sigma B-4501, 99%

### Example 14

Synthesis of Targeting Agent Bio-DLRSGRK (Bio=D-Biotin=Vitamin H), Comprising the Effector Unit D-Biotin Coupled (Linked Directly, Without Specific Linker Units) via its Carboxyl Group to the N-Terminal Amino Group of the Peptide DLRSK by Virtue of an Amide Bond, and also Comprising the Targeting Unit DLRSGRK, that is Cyclic by Virtue Of An Amide Bond Between the Side Chains of Aspartic Acid and Lysine

The targeting agent was synthesized using manual synthesis as described in Example 2 above (analogously to the synthesis in Example 5 above, including cyclization) and using the biotinylation procedure described in Example 13 above as the final coupling step. In this final coupling process, D-biotin was employed instead of a protected amino acid. D-biotin was not protected but was employed as such. The product was isolated and purified in the manner indicated in Example 2 and identified by positive-mode MALDI-TOF spectroscopy (M+1 ion clearly predominant).

MALDI-TOF Data (Bio-DLRSGRK, Cyclic):

calculated molecular mass=1038.54

Observed Signals:

1039.74 M+H

1061.76 M+Na

1077.60 M+K

### Example 15

Synthesis of Targeting Agent Bio-DRGLRSK (Bio=D-Biotin=Vitamin H), Comprising the Effector Unit D-Biotin Coupled (Linked Directly, Without Specific Linker Units) via its Carboxyl Group to the N-Terminal Amino Group of the Peptide DRGLRSK by Virtue of an Amide Bond, and also Comprising the Targeting Unit DRGLRSK, that is Cyclic by Virtue of an Amide Bond Between the Side Chains of Aspartic Acid and Lysine

The targeting agent was synthesized using manual synthesis as described in Example 2 above (analogously to the synthesis in Example 6 above, including cyclization) and using the biotinylation procedure described in Example 13 above as the final coupling step. In this final coupling process, D-biotin was employed instead of a protected amino acid. D-biotin was not protected but was employed as such. The product was isolated and purified in the manner indicated in Example 2 and identified by positive-mode MALDI-TOF spectroscopy (M+1 ion clearly predominant).

MALDI-TOF Data (Bio-DRGLRSK, Cyclic):

calculated molecular mass=1038.56

Observed Signal:

1039.59 M+H

### Example 16

Synthesis of Targeting Agent Bio-AhxDLRSK (Bio=D-Biotin=Vitamin H), Comprising the Effector Unit D-Biotin Coupled (Linked Directly, Without Specific Linker Units) via its Carboxyl Group to the N-Terminal Amino Group of the Peptide AhxDLRSK by Virtue of an Amide Bond, and also Comprising the Targeting Unit DLRSK, that is Cyclic by Virtue of an Amide Bond Between the Side Chains of of the Outermost Members of the Sequence

The targeting agent was synthesized using manual synthesis as described in Example 2 above (analogously to the synthesis in Example 7 above, including cyclization) and using the biotinylation procedure described in Example 13 above as the final coupling step. In this final coupling process, D-biotin was employed instead of a protected amino acid. D-biotin was not protected but was employed as such. The product was isolated and purified in the manner indicated in Example 2 and identified by positive-mode MALDI-TOF spectroscopy (M+1 ion clearly predominant).

MALDI-TOF Data (Bio-AhxDLRSK Cyclic):

calculated molecular mass=938.50

Observed Signal:

939.50 M+H

### Example 17

Synthesis of Targeting Agent Bio-K-AhxDLRSK (Cyclic by Virtue of an Amide Bond Between the Side Chains of Aspartic Acid and C-Terminal Lysine; Bio=D-Biotin=Vitamin H), Comprising One Effector Unit D-Biotin Coupled (Linked via One Plus One Linker Units and/or Spacer Units and/or as One Larger Spacer and/or Linker Unit) via its Carboxyl Group to the N-Terminal Amino Group of the Lysine Residue (Unit) and this in Turn by Virtue of an Amide Bond to the Amino Group of 6-Aminohexanoic Acid (=Ahx) and this by Virtue of an Amide Bond to the Amino Terminus of the Peptide DLRSK, and also Comprising the Targeting Unit DLRSK

The synthesis was carried out as follows: The fully protected resin-bound cyclized targeting unit (peptide with spacer/linker unit) AhxDLRSK was prepared as described in Example 7 above. Next, the sequence AhxDLRSK was continued with one lysine unit (protected with Fmoc-group on N-terminal amino group and with Boc-group on side branch amino group) by means of the general coupling methods described in Example 2. The reagent used as starting material:

Fmoc-L-Lys(tBoc)-OH

Finally the still resin-bound and fully protected K-AhxDLRSK was biotinylated according to the general method described in Example 13. Purification by HPLC gave 30% of the theoretical as overall yield. Identification of the product:

positive mode MALDI-TOF mass spectrum: M+1 ion clearly predominant.

MALDI-TOF Data (Bio-K-AhxDLRSK, Cyclic):

calculated molecular mass=1066.60

Observed Signal:

1067.5 M+H

### Example 18

Synthesis of Targeting Agent Bio₄-K₃-K-AhxDLRSK (Cyclic by Virtue of an Amide Bond Between the Side Chains of Aspartic Acid and C-Terminal Lysine; Bio=D-Biotin=Vitamin H), Comprising Four Identical Effector Units D-Biotin Coupled (Linked via a Dendrimeric Structure that can be Considered as a Combination of Linker Units and/or Spacer Units and/or as One Larger Spacer and/or Linker Unit) Each via its Carboxyl Group to One Amino Group of a Lysine Residue (Unit), Either the N-Terminal Amino Group or the Side-Chain Amino Group, and the Dendrimeric Structure (Two Lysines Each Carrying Two Effector Biotin Units, These Lysines Being Coupled via the Carboxyl Functions to One Further Lysine and this in Turn by Virtue of an Amide Bond to the N-Terminal Amino Group of One Lysine (Having the Side Chain Uncoupled) that is Similarly Linked to Ahx (6-Aminohexanoic Acid) and this by Virtue of an Amide Bond to the Amino Terminus of the Peptide DLRSK, and also Comprising the Targeting Unit DLRSK

The product has the formula shown below: 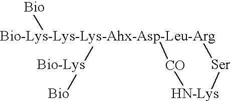
and can be stated to comprise a four-fold biotinylated four-branch linker/spacer unit on the N-terminus of K-AhxDLRSK.

The synthesis was carried out as follows: The fully protected resin-bound, ‘on resin’ cyclized targeting unit (peptide with two spacer/linker units) K-AhxDLRSK was prepared as described in Example 16 above. The branched structure comprising the four biotins and the three lysines was conctructed by means of the general coupling methods described in Example 2, so that the sequence K-AhxDLRSK was continued first with one lysine unit (protected with one Fmoc-group on each of its two amino groups). Then, the procedure (lysine addition) was repeated using doubled amounts of coupling reagents and the doubly protected (Fmoc groups) lysine, in order to couple two more lysine units, one of them on the side-chain amino and one on the amino-terminal amino group of the first-coupled lysine unit. Reagent used (in addition to the materials described in the referred Examples):

Fmoc-L-Lys(Fmoc)-OH, CAS No. 78081-87-5, Molecular weight: 590.7 g/mol, PerSeptive Biosystems Cat. No. GEN911095, Hamburg, Germany

Biotinylation was done according to the general method described in Example 13 using 12 molecular equivalents of coupling reagents and biotin, employing the resin-bound branched peptide, to afford a stucture comprising four biotin units. Purification by HPLC gave 44% of the theoretical as overall yield.

Identification of the Product:

positive mode MALDI-TOF mass spectrum: M+1 ion clearly predominant.

MALDI-TOF Data (Bio₄-K₃-K-AhxDLRSK, cyclic):

calculated molecular mass=2129.12

Observed Signal:

2129.89 M+H (the strongest isotopomer is 2130.9)

### Example 19

Synthesis of Targeting Agent Bio₄-K₃-K(DTPA)-AhxDLRSK (Cyclic by Virtue of an Amide Bond Between the Side Chains of Aspartic Acid and C-Terminal Lysine; Bio=D-Biotin=Vitamin H; DTPA=Diethylenetriaminepentmcetic Acid Minus One OH), Comprising Two Types of Effector Units: Four Identical Effector Units D-Biotin Coupled (Linked via a Dendrimeric Structure that can be Considered as a Combination of Linker Units and/or Spacer Units and/or as One Larger Spacer and/or Linker Unit) Each via its Carboxyl Group to One Amino Group of a Lysine Residue (Unit), Either the N-Terminal Amino Group or the Side-Chain Amino Group, and the Dendrimeric Structure (Two Lysines Each Carrying Two Effector Biotin Units, These Lysines Being Coupled via the Carboxyl Functions to one Further Lysine and this in Turn by Virtue of an Amide Bond to the N-Terminal Amino Group of One Lysine (Having the Side Chain Coupled via Amide Bond to DTPA) that is Similarly Linked to Ahx (6-Aminohexanoic Acid) and this by Virtue of an Amide Bond to the Amino Terminus of the Peptide DLRSK, and also Comprising The Targeting Unit DLRSK

The product has the formula shown below: 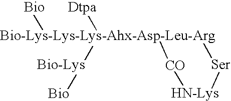
and can be stated to comprise a four-fold biotinylated five-branch linker/spacer unit, carrying Dtpa-moiety on one branch, on the N-terminus of peptide AhxDLRSK.

The synthesis was carried out as follows: The isolated and purified targeting agent Bio₄K₃-K-AhxDLRSK was prepared as described in Example 18 above. The product thus obtained was then treated with 10 molecular equivalents of diethylenetriaminepentaacetic dianhydride in DMF solution (0.01 M solution as calculated on the basis of the biotinylated peptide) for 18 hours. After this treatment, the volume was doubled by addition of water to the DMF solution, and the solution was put aside and allowed to stay still for 4 hours. Finally, the solvents were evaporated in vacuo and the residue was mixed in water containing 0.1% trifluoroacetic acid and was filtered and the filtrate was purified by reversed-phase HPLC. The product was identified by its M+1 peak in the MALDI-TOF mass spectrum.

Identification of the Product:

positive mode MALDI-TOF mass spectrum: M+1 ion clearly predominant.

MALDI-TOF Data [Bio₄-K₃-K(Dtpa)-AhxDLRSK, Cyclic]:

calculated molecular mass=2504.24

Observed Signal:

2505.29 M+H

### Example 20

Synthesis of Targeting Agent Cbp-DLRSK [Cbp=5-(1-O-Carboranyl)-Pentanoyl], Comprising the Effector Unit 5-(1-O-Carboranyl)-Pentanoic Acid Coupled (Linked Directly, Without Specific Linker Units) via its Carboxyl Group to the N-Terminal Amino Group of the Peptide DLRSK by Virtue of an Amide Bond, and also Comprising the Targeting Unit DLRSK, that is Cyclic by Virtue of Lactam Bridge Between the Side Chains of the Outermost Members of the Sequence

The targeting agent was synthesized using manual synthesis as described in Example 2 above (analogously to the synthesis in Example 4 above, including cyclization). Next, the sequence DLRSK was continued with 5-(1-o-carboranyl)-pentanoic acid by means of the general coupling technique described in Example 2 with the exeption of PyAoP (instead of HBTU) and HOAT (instead of HOBt) and reaction time 4 hours in the treatment step 12 of Example 2.

Reagent Used:

5-(1-o-carboranyl)-pentanoic acid, Katchem, Prague, Czech Republic, F.W. 244.34 g/mol

MALDI-TOF Data (Cbp-DLRSK, Cyclic):

calculated molecular mass=817.60 (basis B10, abund. 20%), 827.57 (basis B11 abund. 80%)

average molecular weight=826.01 g/mol

Observed Signals:

Multiplet with highest peaks at 826.55 and 827.55: M+H

Multiplet with highest peaks at 848.45 and 849.50: M+Na

### Example 21

General Method for the Cyclization of a Peptide, Targeting Unit, Targeting Agent or Targeting Motif in the Form of a Lactam (as Macrolactam; by Virtue of an Amide Bond Between the Side Chains of Lysine and Aspartic Acid that were Included in the Sequence at the Ends of an ‘Intermediary’ Sequence)

The uncyclized, fully protected, resin-bound peptides were prepared manually by means of the general method described in Example 2 above.

Prior to the cyclization, a selective, one-process, dismantling of the side-chain protecting groups of lysine and aspartic acid [the said groups were: 4-methyltrityl on the lysine unit and 2-phenylisopropyl (ester) on the aspartic acid unit] was carried out with diluted TFA (4% in dichloromethane). The cyclization involved a condensation between the side-chain carboxyl group of the aspartic acid unit and the 6-amino group (side-chain amino group) of the lysine unit. Activation was by a PyAOP/HOAt/DIPEA reagent mixture (for details and abbreviation explanation, see below) or, alternatively, by PyAOP/DIPEA. The equipment, common solvents, and practical techniques were similar to those described in Example 2.

The initially fully protected resin-bound peptide (e.g. 0.3 mmol) was shaken under argon atmosphere at room temperature with different solutions (about 10 mL) for the periods of time indicated below, followed by filtration:

1. dichloromethane, for 20 min.

2. 4% (by volume) trifuoroacetic acid in dichloromethane, for 15 min.

3. 0.2 M DIPEA in 1:10 mixture of NMP and dichloromethane, for 3 min.

4. dichloromethane, for 3 min.

5. dichloromethane, for 3 min.

6. dichloromethane, for 3 min.

7. DMF, for 3 min.

8. activation, for 4 hours, according to the description below:

A mixture of PyAOP and HOAt, 3 molecular equivalents of both components (or alternatively PyAOP only) with respect to the resin-bound peptide (thus, 0.9 mmol both) in DMF (7 mL), was shaken with the resin for 1 min without filtration, followed by addition of 6 molecular equivalents of 2 M DIPEA in NMP.

After step 8 above, the procedures continued as described in Example 2, starting from step 13 in it.

The reagents for activation in this type of cyclization were:

PyAOP=7-Azabenzotriazol-1-yloxytris(pyrrolidino)phosphonium hexafluorophosphate, CAS No. 156311-83-0, PE Biosystems Cat. No. GEN076531, Molecular Weight: 521.4 g/mol

HOAt=1-Hydroxy-7-azabenzotriazole, 0.5 M solution in DMF, Applied Biosystems Cat. No. 4330631

DIPEA=N,N-Diisopropylethylamine, 2.0 M solution in N-methylpyrrolidinone Applied Biosystems Cat. No. 401517

For materials in the ‘HBTU and HOBt’ alternative, see the materials indicated in Example 2.

Starting materials for the ‘special’ amino acid units (aspartic acid and lysine), between which the ‘extra’ peptide bond was formed:

Fmoc-Lys(Mtt) Resin, 0.68 mmol/g, Bachem Cat. No. D-2565.0005

Fmoc-Asp(2-phenylisopropyl ester)-OH, Molecular weight: 473.53 g/mol, Bachem Cat. No. B-2475.0005

### Example 22

Synthesis of Targeting Agent Amf-DLRSK (Amf=4-Amino-10-Methylfolic Acyl), Comprising the Effector Unit 4-Amino-10-Methylfolic Acid Coupled (Linked Directly, Without Specific Linker Units) via its Carboxyl Group to the N-Terminal Amino Group of the Peptide DLRSK by Virtue of an Amide Bond, and also Comprising the Targeting Unit DLRSK, that is Cyclic by Virtue of Lactam Bridge Between the Outermost Members of the Sequence

The targeting agent was synthesized using manual synthesis as described in Example 2 above (analogously to the synthesis in Example 4 above, including cyclization). Next, the sequence DLRSK was continued with 4-amino-10-methylfolic acid by means of the general coupling technique described in Example 2 with the exceptions of PyAOP (instead of HBTU) and HOAT (instead of HOBt) and reaction time 5 hours and equimolar ratio of reagents to resin-bound peptide (peptide/PyAOP/HOAT/DIPEA=1:1:1:2) in the treatment step 12 of Example 2.

Reagent Used:

4-amino-10-methylfolic acid hydrate; (+)amethopterin; methotrexate CAS No. 59-05-2, Formula weight: 454.4 g/mol, Sigma Cat. No. A-6770

MALDI-TOF Data (Amf-DLRSK, Cyclic):

calculated molecular mass=1035.50

Observed Signals:

1036.35 M+H

### Example 23

Synthesis of Targeting Agent Dnm-Aoa-DLRSK (Dnm=Daunomycin Linked via its Peripheral Carbonyl Group by Loss of One Oxygen), Comprising the Effector Unit Daunomycin Coupled via its Carbonyl Group at Acetyl Moiety by Oxime Ligation to the Aminooxy Group of Aoa-DLRSK [a Targeting Agent (Derivative of Peptide) Comprising the Linker (Ligation) Unit Aminooxyacetic Acid Coupled (Linked Directly, Without Specific Linker Units) via its Carboxyl Group to the N-Terminal Amino Group of the Peptide DLRSK by Virtue of an Amide Bond], and also Comprising the Targeting Unit DLRSK, that is Cyclic by Virtue of Lactam Bridge

The targeting agent was synthesized by stirring Aoa-DLRSK, described above in Example 10, with equimolar amount of daunomycin hydrochloride in methanol solution (concentration 0.0025M) at room temperature in dark for three days. The product was isolated by evaporation of solvents and purified by reverse phase HPLC as described in Example 2.

Reagent Used:

Daunomycin hydrochloride, CAS No. 20830-81-3, Molecular weight: 564.0 g/mol, ICN Biomedicals, Aurora, Ohio, USA, Cat. No. 44583

MALDI-TOF Data (Dnm-Aoa-DLRSK, Cyclic):

calculated molecular mass=1181.52

Observed Signal:

1182.41 M+H

### Example 24

Synthesis of Targeting Agent Dxrb-Aoa-DLRSK (Dxrb=Doxorubicin Linked via its Peripheral Carbonyl Group by Loss of One Oxygen), Comprising the Effector Unit Doxorubicin Coupled via its Carbonyl Group at Hydroxyacetyl Moiety by Oxime Ligation to the Aminooxy Group of Aoa-DLRSK [A Targeting Agent (Derivative of Peptide) Comprising the Linker (Ligation) Unit Aminooxyacetic Acid Coupled (Linked Directly, Without Specific Linker Units) via its Carboxyl Group to the N-Terminal Amino Group of the Peptide DLRSK by Virtue of an Amide Bond], and also Comprising the Targeting Unit DLRSK, that is Cyclic by Virtue of Lactam Bridge

The targeting agent was synthesized by stirring Aoa-DLRSK, described above in Example 10, with equimolar amount of doxorubicin hydrochloride in methanol solution (concentration 0.0025M) at room temperature in dark for three days. The product was isolated by evaporation of solvents and purified by reverse phase HPLC as described in Example 2.

Reagent Used:

Doxorubicin hydrochloride, CAS No. 25316-40-9, Molecular weight: 580.0 g/mol, Fluka Cat. No. 44583

MALDI-TOF Data (Dxrb-Aoa-DLRSK, Cyclic):

calculated molecular mass=1197.52

Observed Signal:

1198.17 M+H

Structural Formula:

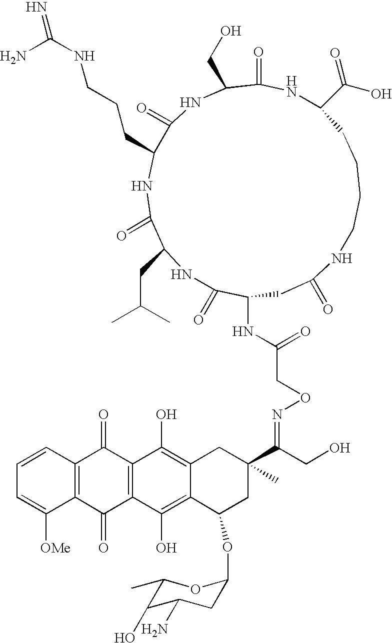

### Example 25

Preparation of Fusion Proteins Comprising a Targeting Unit

Synthetic DNA sequences encoding the desired amino acid sequences were produced by annealing two complementary oligonucleotides (Genset SA) comprising either EcoRI or BamHI restriction sites in their 5′ ends, and a stop codon in the 3′ end of the coding strand, at 65° C. for 1 min. For production of the DNA encoding the targeting peptides, partially overlapping oligonucleotides were used and the double-stranded product was synthesized at 72° C. for 30 s in the presence of free dNTPs.

The following oligonucleotides were used for production of the DNA encoding the different targeting sequences:

GCLRSC:

forward primer:
5′-CGGGATCCGGGTGTCTTCGGAGTTGTTGAGAATTCC-3′;
reverse primer:
5′-GGAATTCTCAACAACTCCGAAGACACCCGGATCCCG-3′
CSRLC:
forward primer:
5′-CGGGATCCTGTAGTCGGCTTTGTTGAGAATTCC-3′;
reverse primer:
5′-GGAATTCTCAACAAAGCCGACTACAGGATCCCG-3′
GLRS:
forward primer:
5′-CGGGATCCGGTTTACGTTCTTGAGAATTCC-3′,
reverse primer:
5′-GGAATTCTCAAGAACGTAAACCGGATCCC-3′
LRS:
forward primer:
5′-CGGGATCCTTACGTTCTTGAGAATTCC-3′,
reverse primer:
5′-GGAATTCTCAAGAACGTAAGGATCCC-3′
GSRL:
forward primer:
5′-CGGGATCCGGTAGTCGGCTTTGAGAATTCC-3′,
reverse primer:
5′-GGAATTCTCAAAGCCGACTACCGGATCCC-3′
SRL:
forward primer:
5′-CGGGATCCAGTCGGCTTTGAGAATTCC-3′,
forward primer:
5′-GGAATTCTCAAAGCCGACTGGATCCC-3′

The double-stranded products were digested with BamHI and EcoRI and the fragments were ligated into the corresponding restriction sites of the pGEX-2TK vector (AmershamPharmacia Biotech). Competent E. coli BL21 bacteria were transformed with the ligation mixture and transformants were screened using colony-PCR (PCR=polymerase chain reaction). Primers specific for the insert-flanking regions of the pGEX vector were used for identification of the inserts (forward primer: 5′-GCATGGCCTTTGCAGGG-3′; reverse primer: 5′-AGCTGCATGTGTCAGAGG-3′). DNA was isolated from positive clones using a QIAprep Spin miniprep kit (cat. no. 27106; Qiagen).

The DNA sequence of the constructs was determined with an ALF automated DNA sequencer (AmershamPharmacia Biotech) using the same primers as for the colony-PCR. Large scale production and purification of GST and of GST-fusion proteins was done according to AmershamPharmacia's instructions (GST detection module instructions, Technical document XY0460012-Rev.8.pdf; Uppsala, Sweden). The size, quantity and purity of the GST-fusion proteins were examined by SDS-PAGE (=sodium-dodecylsulphate polyacrylamide gel electrophoresis).

### Example 26

In Vivo Targeting of Tumors in Mice

In this example in vivo targeting of the targeting units prepared in the previous examples is shown for four different types of primary tumors (fibrosarcoma, Kaposi's sarcoma, melanoma, gliablastoma and adenocarcinoma) and for melanoma metastases in lung. It is shown that the tested targeting units according to the present invention selectively target to primary tumors and to metastases in vivo but not to normal tissues or organs.

Cell Lines and Tumor-Bearing Mice

The following tumor cell lines were used to produce experimental tumors in mice:

“ODC sarcoma cells”, (OS), originally derived from tumors that were formed in nude mice to which had been administered NIH3T3 mouse fibroblasts transformed by virtue of ornithine decarboxylase (ODC) overexpression and have been described earlier (Auvinen et al., 1992);

Kaposi's sarcoma cell line, KS1767, (KS), described previously (Herndier et al., 1996);

A human melanoma cell line C8161 (M) described by Welch et al. (1991);

A glioblastoma cell line U-87 MG, ATCC HTB14, (GB) previously described (Beckman et al. 1971, Fogh et al. 1977); and

Non-small cell lung cancer line NCI-H23, ATCC No. 5800, (AC) described previously (Mase et al., 2002).

The cell lines were cultured in Dulbecco's Modified Eagle's Medium (DMEM; Bio-Whittaker) supplemented with 5-10% fetal calf serum (FCS; Bio-Whittaker), 1% L-glutamine (Bio-Whittaker) and 1% penicillin/streptomycin (Bio-Whittaker). The U-87 MG cell line was cultured in Minimum essential medium Eagle with 2 mM L-glutamine and Earle's BSS adjusted to contain 1.5 g/l sodium bicarbonate, 0.1 mM non-essential amino acids, 1.0 mM sodium pyryvate, and 10% fetal bovine serum. The NC1-H23 cell line was cultured in RPMI 1640 medium with 2 mM L-glutamine adjusted to contain 1.5 g/L sodium bicarbonate, 4.5 g/L glucose, 10 mM HEPES, and 1.0 mM sodium pyruvate, 90%; fetal bovine serum, 10%.

Experimental Tumor Production

For production of experimental tumors, the cells listed above (OS, KS and melanoma: 0.5×106 cells, U-87 MG: 1×106 cells and NCI-H23 3×106 cells) were injected subcutaneously into both flanks of nude mice of the strains Balb/c Ola Hsd-nude, NMRI/nu/nu or Athymic-nu (all mice of both strains were from Harlan Laboratories). Tumors were harvested when they had reached a weight of about 0.4 g.

Metastases (mostly formed in the lungs) were produced by injection of melanoma cells i.v. into Balb/c Ola Hsd-nude mice. The mice were kept 4-6 weeks, and then targeting experiments were performed.

Tumor-bearing or metastase-bearing mice were anesthesized by administering 0.02 ml/g body weight Avertin [10 g 2,2,2-tribromoethanol (Fluka) in 10 ml 2-methyl-2-butanol (Sigma Aldrich)] intraperitoneally (i.p.).

In Vivo Targeting and Detection of Targeting

For localization of the targeting peptides KS, OS or melanoma tumor-bearing or metastase-bearing NMRI nude mice were anesthesized and 1 or 2 mg of GST-fusion proteins prepared in Example 25 in DMEM, or GST alone in DMEM as control, was injected intravenously or intraperitoneally. Alternatively, either 1 or 2 mg of biotinylated synthetic targeting peptide (prepared in Example 12) was injected i.v. 5-10 min after the i.v. injections, the mice were perfused via the heart using a winged infusion 25 G needle set (Terumo) with 50 ml DMEM. Then, their organs were dissected and frozen in liquid nitrogen. In some cases, a GST-fusion protein was injected i.v. as above, and then the mice were sacrified after 30 min, 4 h, 8 h or 18 h, without perfusion, and then tumors and control organs (liver, kidney, spleen, heart, brain) were dissected and frozen in liquid nitrogen. Intraperitoneally injected mice were kept 24 h before sacrification, and then tumors and control organs were dissected and frozen as above.

The GST-fusion proteins (and GST as control) were detected on 10 micrometer cryosections by goat anti-GST antiserum (AmershamPharmacia).

Biotinylated peptides/peptidomimetic analogues/peptidyl analogues (targeting agents) were detected on 10 micrometer cryosections using AB (avidin-biotin)-complex containing avidin, and biotinylated HRP (Vectastain ABC-kit, cat no. PK6100; Vector Laboratories) with diaminobenzidine (DAB substrate kit, cat no. 4100, Vector Laboratories).

The results of the experiments are shown in Table 2.

TABLE 2

| | | targeting | tumor | | | | | | |
|---|---|---|---|---|---|---|---|---|---|
| Targeting agent | dose | time | type | tumor | liver | kidney | spleen | heart | brain |
| GST-GCLRSC | 1 mg i.v. | 10 min | OS | + | − | − | − | − | − |
| GST-GCLRSC | 2 mg i.p. | 24 h | KS | + | − | − | − | − | − |
| GST-GCLRSC | 2 mg i.p. | 24 h | OS | + | − | − | − | − | − |
| GST-GCLRSC | 2 mg i.v. | 8 h | M-met | + | − | − | − | − | − |
| GST-GCLRSC | 2 mg i.v. | 18 h | M | + | − | − | − | − | − |
| GST-GCLRSC | 1 mg i.v. | 10 min | AC | + | − | − | − | − | − |
| GST-GCLRSC | 1 mg i.v. | 10 min | GB | + | − | − | − | − | − |
| GST-CSRLC | 1 mg i.v. | 10 min | OS | + | − | − | − | − | − |
| GST-CSRLC | 1 mg i.v. | 10 min | M | + | − | − | − | − | − |
| Bio-DLRSK | 1 mg i.v. | 10 min | OS | + | − | − | − | − | − |
| Bio-DLRSK | 1 mg i.v. | 10 min | M | + | − | − | − | − | − |

### Example 27

Therapeutic Effect of Targeting Agent Comprising Cytotoxic Effector Unit

In this experiment the targeting agent, Dxrb-Aoa-DLRSK prepared in Example 24, comprising a cytotoxic effector unit, doxorubicin, linked by oxime ligation to the cyclic targeting unit DLRSK comprising the targeting motif LRS was used to demonstrate in vivo targeting and therapeutic effect on melanoma tumors.

1 million C8161M/T1 melanoma cells were injected subcutaneously into flank of eight Athymic-nu mice and tumours were allowed to grow for one week. The mice were then divided into three groups those received the following treatments:
Group DMEM: two mice, DMEM only
Group Dox: two mice, 1,43 mg/kg doxorubicin dissolved in DMEM
Group pept+dox: four mice, 1,43 mg/kg Dxrb-Aoa-DLRSK (doxorubicin linked to targeting mofif LRS) dissolved in DMEM (dose equimolar to Group Dox)

Treatments were administered i.v. twice a week (Tuesdays and Fridays), total of five doses were injected. Tumours were measured with a calliper in two perpendicular directions on each injection day and on the day the animals were sacrificed. Tumour volume was calculated by the formula for ellipsoid:
Volume=(length×width²)×0.5

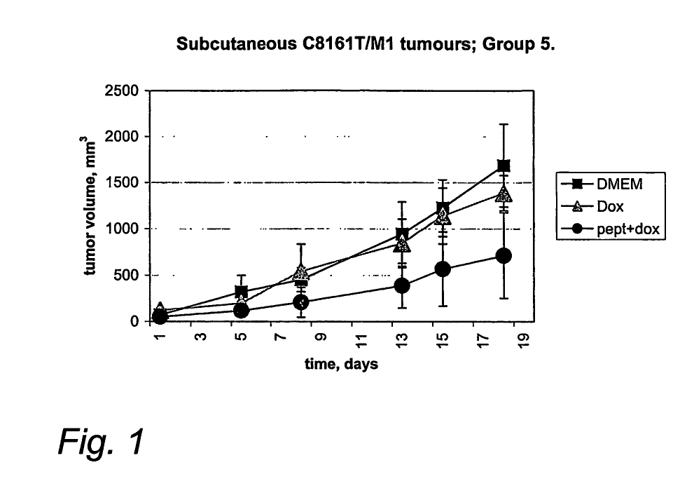

The result of the experiment is shown in FIG. 1 and confirms that a targeting agent according to the present invention selectively targets to melanoma tumor in vivo and significantly increases the therapeutic effect of doxorubicin.

Reagent Used:

Doxorubicin hydrochloride, CAS No. 25316-40-9, Molecular weight: 580.0 g/mol, Fluka Cat. No. 44583

### Example 28

General Method for the Cyclization of a Peptide or Related Substance by Virtue of an Amide Bond Between D-Ornithine and Glutamic Acid Those are Included in the Sequence at the Ends of an ‘Intermediary’ Sequence: Formation of ‘Head-To-Side-Chain Macrolactam’, ie., ‘Glu(D-Orn)-Ring’

The uncyclized, fully protected, resin-bound peptides are prepared manually by means of the general method described above.

Prior to the cyclization, a selective, one-process, dismantling of particular protecting groups of ornithine and gutamic acid [the said groups are: 2-N-Fmoc on the ornithine unit and 5-(2-trimethylsilylethyl ester) on the glutamic acid unit] is carried out with tetrabutylammonium fluoride solution in DMF. The cyclization involves a condensation between the side-chain carboxyl group of the glutamic acid unit and the 2-amino group (N-terminal amino group) of the ornithine unit. Activation is by a PyAOP/DIPEA reagent mixture (for details and abbreviation explanation, see below) instead of the HBTU/HOBt/DIPEA mixture described in Example 2. The equipment, common solvents, and practical techniques are similar to those described in Example 2.

This method can be modified for lysine (instead of ornithine) and aspartic (instead of glutamic) acid unitst by empoying respective derivatives of those amino acids.

The initially fully protected resin-bound peptide (0.3 mmol) is shaken under argon atmosphere at mom temperature with different solutions (about 10 mL) for the periods of time indicated below, followed by filtration:
1. Dichloromethane, for 20 min.
2. 1 M tetrabutylammonium fluoride in DMF, for 20 min.
3.-5. DMF, for 1 min (three treatments).
6.-8. DCM, for 1 min (three treatments).
9. DMF, for 1 min.
10. 0.9 mmol of PyAOP (3 molecular equivalents with respect to the resin-bound peptide) in DMF (7 mL), is shaken with the resin for 1 min without filtration.
11. Addition of 6 molecular equivalents of 2 M DIPEA (thus, 1.8 mmol) in NMP, followed by shaking for 4 hours.

After the steps above, the resin is washed etc. as described in the general procedure for (manual) peptide synthesis (the steps after addition of activated amino acid).

The reagent for deprotection prior to cyclization is: Tetrabutylammonium fluoride trihydrate, CAS No. 87749-50-6, molecular weight: 315.51 g/mol, Acros Organics Cat. No. 221080500.

The reagents for activation in this type of cyclization are:

PyAOP=7-Azabenzotriazol-1-yloxytris(pyrrolidino)phosphonium hexafluorophosphate, CAS No. 156311-83-0, PE Biosystems Cat. No. GEN076531, Molecular Weight: 521.4 g/mol

DIPEA=N,N-Diisopropylethylamine, 2.0 M solution in N-methylpyrrolidinone, Applied Biosystems Cat. No. 401517

Starting materials for the ‘special’ amino acid units (glutamic acid and ornithine), between which the ‘extra’ amide bond is formed:

Fmoc-D-Orn(Mtt)-OH; 2-N-Fmoc-5-N-(4-methyltrityl)-D-ornithine, molecular weight: 610.8 g/mol, Novabiochem Cat. No. 04-13-1012.

Fmoc-L-Glu(OTMSEt)-ONa; N-2-Fmoc-glutamic acid 5-(2-trimethylsilylethyl) ester sodium salt, molecular weight: 468.60 g/mol, Novabiochem Cat. No. 04-12-1231.

### Example 29

Synthesis of Targeting Unit (Peptide) D-OrnLRSE-Amide, Cyclic by Virtue of an Amide Bond Between the Side Chain of Glutamic Acid Unit and the α-Amino Group of D-Ornithine

The functionally protected, resin bound targeting unit (protected peptide), comprising targeting motif LRS, was synthesized by means of manual synthesis as described in Example 2 above, in which the the “empty” resin was deprotected prior to the first coupling in the same manner as described for the the pre-loaded resins (steps 1-11 in Example 2).

The following reagents were employed as starting materials (in this order):

Rink amide MBHA Resin

Fmoc-L-Glu(OTMSEt)-OH

Fmoc-L-Ser(tBu)-OH

Fmoc-L-Arg(Pbf)-OH

Fmoc-L-Leu-OH

Fmoc-D-Orn(Mtt)-OH

After the last cycle of the coupling process, the still resin-bound targeting unit was subjected to the cyclization process in which an extra amide bond is formed as described in Example 28. Next, a sample of peptide was cleaved from the resin by three hours' treatment with the cleavage mixture described in Example 2, and isolated as described in the same example.

Then, the product was identified with the aid of its positive mode MALDI-TOF mass spectrum, in which the M+1 ion of cyclic D-OrnLRSE-amide was clearly predominant.

MALDI-TOF data (Cyclic D-OrnLRSE-NH₂):

calculated molecular mass=598.36

Observed Signal:

599.42 M+1

### Example 30

Synthesis of Targeting Agent Cptc-AhxDLRSK [Cptc=(S)-(+)-Camptothecin Linked as Ester at its Hydroxyl Group via Carbonic Acyl, i.e. (S)-(+)-Camptothecin Carbonyl Moiety], Comprising the Effector Unit Camptothecin Carbonate Coupled to the Amino Group at 6-Aminohexanoyl (=Ahx) Moiety of The Peptide AhxDLRSK by Virtue of an Amide Bond (or Targeting Agent Where Effector Unit (S)-(+)-Camptothecin is Linked via the Spacer Unit 6-(Carbonylamino)-Hexanoyl to the Targeting Unit DLRSK), and also Comprising the Targeting Unit DLRSK, that is Cyclic by Virtue of an Amide Bond Between the Side Chains of the Outermost Members of the Sequence

Camptothecin p-nitrophenylcarbonate, described in the end of this example, was dissolved as 0.02 M solution in DMF and combined with 0.04 M solution of equimolar amount of cyclic targeting compound AhxDLRSK, described in Example 7 above, in the same solvent. After staying overnight, 2 M DIPEA in NMP was added in 10% excess (i.e. equimolar amount multiplied by 1.1). After being stirred overnight the mixture was diluted with diethyl ether and the centrifuged solid precipitate was purified by reverse phase HPLC chromatography as described in Example 2, including the identification of the product based on its M+1 ion in the positive mode MALDI-TOF mass spectrum.

MALDI-TOF Data (Cptc-AhxDLRSK, Cyclic):

Calculated molecular mass=1086.51

Observed Signal:

1087.26 M+1

The synthesis of camptothecin p-nitrophenylcarbonate: 0.29 mmol of 4-nitrophenyl chloroformate and 0.10 mmol of (S)-(+)-camptothecin were dissolved in 12 mL of dichloromethane (DCM). Next, 1.71 mmol of 4-(dimethylamino)-pyridine (DMAP) was added to the DCM solution on cooling water bath. The mixture was then shaken for two hours followed by dilution with 30 mL of DCM. After washings: twice with 0.1% hydrochloric acid and once with saturated aqueous sodium chloride solution, the DCM solution was dried with disodium sulfate, filtered, and concentrated to small volume. The product was precipitated by addition of diethyl ether and gathered after centrifugation.

Materials used in the synthesis of camptothecin p-nitrophenylcarbonate:

4-nitrophenyl chloroformate, CAS No. 7693-46-1, molecular weight: 201.57 g/mol, Fluka product No. 23240.

(S)-(+)-camptothecin, CAS No. 7689-03-4, molecular weight: 348.36 g/mol, Aldrich product No. 36,563-7.

DMAP; 4-(dimethylamino)-pyridine, CAS No. 1122-58-3, molecular weight 122.17, Fluka product No. 29224.

### Example 31

Synthesis of Targeting Agent D-Orn(Dota)LRSE-AMIDE (Dota=1,4,7,10-Tetraazacyclododecane-1,4,7,10-Tetraacetic Acid Coupled by its One Carboxyl), Cyclic by Virtue of an Amide Bond Between the Side Chain of Glutamic Acid Unit And The α-Amino Group of D-Ornithine

The functionally protected, resin-bound, and cyclized targeting unit, comprising the targeting motif LRS, was synthesized by means of manual synthesis as described in Example 29 above. Next, the resin was treated with diluted TFA (4% in dichloromethane) in the manner described in example 21 (steps 1-7) to cleave the side chain protecting Mtt-group of ornithine. The still resin-bound unit was then coupled with DOTA-tris-tert-butyl ester by means of the general method described in Example 2 (steps 12-18) using HBTU/HOBt/DIPEA activation. Reagent used:

DOTA-tris-(tBu ester).

The product was cleaved and isolated as described in Example 2 and identified with the aid of its positive mode MALDI-TOF mass spectrum, in which the M+1 ion of cyclic D-Orn(Dota)LRSE-amide was clearly predominant.

MALDI-TOF data [cyclic D-Orn(Dota)LRSE-NH₂]:

calculated molecular mass=984.54

Observed Signal:

985.52 M+1

### Example 32

Synthesis of Targeting Unit (Peptide) KLRSD-Amide, Cyclic by Virtue of an Amide Bond Between the Side Chain of Aspartic Acid Unit and the α-Amino Group of Lysine

The functionally protected, resin bound targeting unit (protected peptide), comprising the targeting motif LRS, was synthesized by means of manual synthesis as described in Example 2 above, in which the the “empty” resin was deprotected prior to the first coupling in the same manner as described for the the pre-loaded resins (steps 1-11 in Example 2).

The following reagents were employed as starting materials (in this order):

Rink amide MBHA Resin

Fmoc-L-Asp(OTMSEt)-OH

Fmoc-L-Ser(tBu)-OH

Fmoc-L-Arg(Pbf)-OH

Fmoc-L-Leu-OH

Fmoc-L-Lys(Mtt)-OH

After the last cycle of the coupling process, the still resin-bound targeting unit was subjected to the cyclization process in which an extra amide bond is formed as described in Example 29 (as modification which replaces Glu with Asp and Lys with Orn). Next, a sample of peptide was cleaved from the resin by three hours' treatment with the cleavage mixture described in Example 2, and isolated as described in the same example.

Then, the product was identified with the aid of its positive mode MALDI-TOF mass spectrum by means of M+1 ion.

MALDI-TOF Data (Cyclic KLRSD-NH₂):

calculated molecular mass=598.36

Observed Signal:

599.21 M+1

### Example 33

Synthesis of Targeting Agent K(Dota)LRSD-Amide (Dota=1,4,7,10-Tetraazacyclododecane-1,4,7,10-Tetraacetic Acid Coupled by its One Carboxyl), Cyclic by Virtue of an Amide Bond Between the Side Chain of Asparic Acid Unit and the α-Amino Group of Lysine

The functionally protected, resin-bound, and cyclized targeting unit, comprising the targeting motif LRS, was synthesized by means of manual synthesis as described in Example 32 above. Next, the resin was treated with diluted TFA (4% in dichloromethane) in the manner described in example 21 (steps 1-7) to cleave the side chain protecting Mtt-group of lysine. The still resin-bound unit was then coupled with DOTA-tris-tert-butyl ester by means of the general method described in Example 2 (steps 12-18) using HBTU/HOBt/DIPEA activation.

Reagent used: DOTA-tris-(tBu ester).

The product was cleaved and isolated as described in Example 2 and identified with the aid of its positive mode MALDI-TOF mass spectrum by means of M+1 ion.

MALDI-TOF Data [Cyclic K(Dota)-LRSE-NH₂]:

calculated molecular mass=984.54

Observed Signal:

985.52 M+1

### Example 34

Synthesis of Targeting Unit Ac-DLRSK-Ahx, Cyclic via Side Chains of Aspartic Acid and Lysine

The preparation of Ac-DLRSK-Ahx was executed by manual solid phase peptide synthesis technique that is described in details in Example 2.

The binding of the first structural component (moiety), 6-aminohexanoic acid (=Ahx) whose amino function was protected by 9-fluorenylmethyloxycarbonyl group (=Fmoc group), to a hydroxyl-functionalized peptide synthesis resin was carried out by means of dichlorobenzoyl chloride method (the “equivalents” below are molecular or “mol” amounts relative to the loading capacity of the resin):

The unloaded (“empty”) resin was first washed by shaking with N,N-dimethylformamide (=DMF) for 20 min and filtered. After addition of five equivalents of the Fmoc-protected 6-aminohexanoic acid (Fmoc-Ahx-OH) in DMF (0.2 M solution) and eight equivalents of pyridine onto the resin it was shaked for 3 min. Next, five equivalents of 2,6-dichlorobenzoylchloride was added and the mixture was shaken for 18 h (overnight).

After the lengthy treatment the resin was filtered and washed several times with DMF and dichloromethane in the way described in Example 2 (steps 13-18). Next, the resin was shaken for 2 hours with a mixture of acetic anhydride (2M solution, 94 equivalents) and N,N-diisopropylethylamine (DIPEA, 1.6 M solution, 80 equivalents) in N-methyl pyrrolidinone (NMP) solution, filtered and washed like earlier ending up in drying at argon gas flow.

The reagents used this far were:

HMP Resin, loading capacity: 1.16 mmol/g, Applied Biosystems Cat. No. 400957.

2,6-dichlorobenzoyl chloride, CAS No. 225-102-4, molecular weight: 209.46 g/mol, Lancaster (Morecambe, England), Cat. No. 8922.

Pyridine, Merck Art. No. 9728.

Fmoc-6-aminohexanoic acid (Fmoc-Ahx-OH), CAS No. 88574-06-5, Novabiochem Cat. No. 04-12-1111, Molecular Weight: 353.4 g/mol.

Acetic anhydride, Fuka Cat. No. 45830.

From this on, the synthesis proceeds according to the general method decribed in Example 2. The stuctural reagents used next in this synthesis, are in sequence as follows:

Fmoc-Lys(Mtt)-OH

Fmoc-L-Ser(tBu)-OH

Fmoc-L-Arg(Pbf)-OH

Fmoc-L-Leu-OH

Fmoc-Asp(2-phenylisopropyl ester)-OH

The still resin-bound product was next cyclized according to Example 21. Finally the sequence was continued with acetic acid (i.e. end-capped at amino terminal) as follows: Amino protecting Fmoc-group was removed as described in Example 2 (steps 1-10). Then the still resin-bound product was treated with the mixture of acetic anhydride and DIPEA in NMP like was done after the initial binding of Ahx moiety to the resin. In the end the product was released from the resin and purified as described in Example 2. Identification was based on M+1 ion of MALDI mass spectrum.

MALDI-TOF Data (Cyclic Ac-DLRSK-Ahx)

calculated molecular mass=754.43

observed signal: 755.60

### Example 35

Synthesis of Targeting Agent Ac-DLRSK-Ahx-Dox (Dox=Doxorubicin Coupled via its Amino Group) Compricing Doxorubicin Linked via an Amide Bond to the Carboxyl Group of C-Terminal Spacer Mioety (Ahx=6-Aminohexanoyl) of the N-Capped (Ac=Acetyl) Cyclic Targeting Unit Ac-DLRSK-Ahx Compricing Targeting Motif LRS

The “targeting unit” compound (a peptide derivative) Ac-DLRSK-Ahx was prepared as described in Example 34. Doxorubicine was linked to purified Ac-DLRSK-Ahx in N,N-dimethylformamide (=DMF) solution by means of PyAOP/DIPEA activation as follows:

Equimolar amounts of Ac-DLRSK-Ahx and PyAOP were combined in DMF as 0.05 M solution, two molar equivalents of DIPEA (2 M solution in NMP) was mixed in and after five minutes equimolar (in respect to Ac-DLRSK-Ahx) amount of doxorubicin hydrochloride (0.05 M solution in DMF) was added. After the reaction was allowed to proceed one hour at dark (protected from light) the mixture was diluted with diethyl ether. The centrifugued solid precipitate was purified by reverse phase HPLC chromatography and identified by positive mode MALDI mass spectrum as described in Example 2.

Formula of Ac-DLRSK-Ahx-Dox:

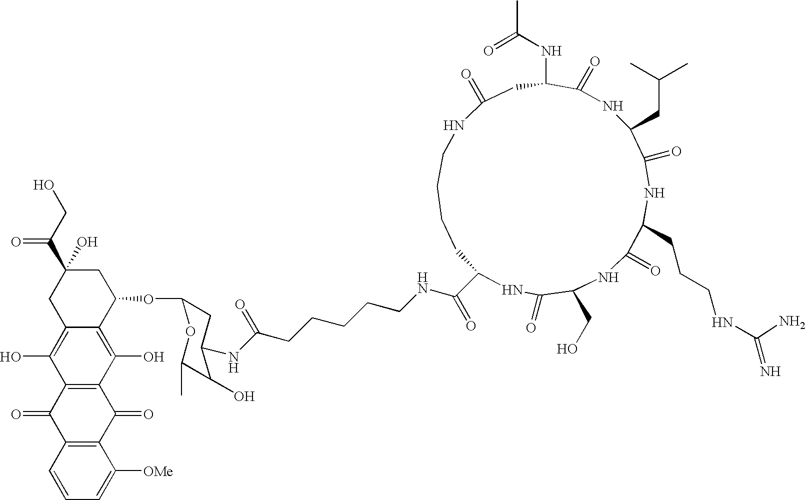

Material Used:

Doxorubicin hydrochloride, CAS No. 25316-40-9, molecular weight: 580.0 g/mol, Sigma Cat. No. D-1515.

MALDI-TOF Data (Ac-DLRSK-Ahx-Dox, -DLRSK-Moiety Cyclic):

calculated molecular mass=1279.60

Observed Signal:

1280.29 M+1

### Example 36

Synthesis of Targeting Agent Amf-AhxDLRSK [Amf=4-Amino-10-Methylfolic Acyl], Comprising the Effector Unit 4-Amino-10-Methylfolic Acid Coupled via its Carboxyl Group to the N-Terminal Amino Group of the Peptide AhxDLRSK by Virtue of an Amide Bond, and also Comprising the Targeting Unit DLRSK, that is Cyclic by Virtue of Lactam Bridge Between the Side Chains of the Outermost Members of the Sequence.

The resin-bound, a spacer (=Ahx) comprising targeting unit (AhxDLRSK) was prepared as described in Example 7 above, including cyclization (according to Example 21). Next, the sequence AhxDLRSK was continued on resin with glutamic acid by means of the general coupling technique described in Example 2. As final coupling the sequence (now E-AhxDLRSK, where “E” will be a part of the “Amf” moiety) was ended with “Amf minus E acid”, i.e. 4-[N-(2,4-diamino-6-pteridinyl-methyl)-N-methylamino]-benzoic acid hemihydrochloride dihydrate, by means of the general coupling techniques with the exceptions of PyAOP as activation reagent (instead of HBTU and HOBt), reaction time 5 hours, and nealy equimolar ratio of reagents to resin-bound peptide in the treatment step 12 of Example 2.

The stoichiometric reagent ratios in that step were: “resin-bound peptide”/“Amf minus E acid”/PyAOP/DIPEA=1:1.2:1.2:2.4, (time 5 h).

After isolation and purification, according to Example 2, the product was identified on the basis of M+1 ion in positive mode MALDI mass spectrum.

Reagents Used:

Fmoc-L-Glu(OtBu)-OH, CAS No. 71989-18-9, Applied Biosystems Cat. No. GEN911036, Molecular Weight: 425.5 g/mol.

4-[N-(2,4-diamino-6-pteridinyl-methyl)-N-methylamino]-benzoic acid hemihydrochloride dihydrate, CAS No. 19741-14-1, Aldrich Cat No. 86,155-3, molecular weight: 379.59 g/mol, designated as “Amf minus E acid”.

MALDI-TOF Data (Amf-AhxDLRSK, Cyclic):

calculated molecular mass=1148.58

Observed Signals:

1149.62 M+H

### Example 37

Synthesis of Targeting Agent PtxSuc-AhxDLRSK (PtxSuc=Paclitaxel Monosuccinate), Comprising the Effector Unit Paclitaxel as Monosuccinate Coupled via its Succinyl (Succinic Carboxyl) Group to the Amino Group at 6-Aminohexanoyl (=Ahx) Moiety of the Peptide AhxDLRSK by Virtue of an Amide Bond (or Targeting Agent Where Effector Unit Paclitaxel is Linked via the Spacer Unit 6-(Succinylamino)-Hexanoyl to the Targeting Unit DLRSK), and also Comprising the Targeting Unit DLRSK, that is Cyclic by Virtue of an Amide Bond Between the Side Chains of the Outermost Members of the Sequence

Paclitaxel succinate, described in the end of this example, was dissolved as 0.012 M solution in DMF and equimolar amount of 0.05 M PyAOP in DMF was added, followed by double molar amount of 2.0 M DIPEA in NMP. After 2 minutes equimolar amount (per paclitaxel succinate) of side-chain-to-side-chain cyclic targeting compound AhxDLRSK, described in Example 7 above, was added as 0.015 M solution in DMF. After staying overnight the mixture was diluted with diethyl ether. The centrifuged solid precipitate was purified by reverse phase HPLC chromatography as described in Example 2, including the identification of the product based on its M+1 ion in the positive mode MALDI-TOF mass spectrum.

MALDI-TOF Data (PtxSuc-AhxDLRSK, Cyclic):

calculated molecular mass=1647.76

Observed Signal:

1648.57 M+1

Paclitaxel succinate was synthesized by following the procedure described in the article: Chun-Ming Huang, Ying-Ta Wu and Shui-Tein Chen (2000). Targeting delivery of paclitaxel into tumor cells via somatostatin receptor endocytosis. Chemistry & Biology 2000, Vol 7 No 7. 453-461.

Herewith 0.05 M paclitaxel in pyridine was stirred with 12-fold excess of succinic anhydride for 3 hours. After evaporation of the solvent in reduced pressure, the residue was dissolved in water and freeze-dryed (lyophilized).

Materials used in the synthesis of paclitaxel succinate:

Paclitaxel (from Taxus yannesis), CAS No. 33069-62-4, molecular weight: 853.9 g/mol, Sigma product No. T-1912.

Succinic anhydride, CAS No. 108-30-5, molecular weight: 100.08 g/mol, Fuka product No. 14089.

List of Reagents

Acetic anhydride, CAS No. 108-24-7, Molecular weight: 102.1 g/mol, Fluka product No. 45830

4-Amino-10 methylfolic acid; (+)amethopterin; methotrexate hydrate; Formula weight: 454.4 g/mol, CAS No. 59-05-2, Sigma A-6770

Boc-amino-oxyacetic acid; Boc-NH—OCH2-COOH, Molecular weight: 191.2 g/mol, Novabiochem Product No. 01-63-0060

Boc-Cys (Trt)-OH, CAS No: 21947-98-8, Novabiochem, product no 04-12-0020

D-Biotin (Vitamin H), CAS No. 58-85-5, molecular weight: 244.3 g/mol, Sigma B-4501, 99%

(S)-(+)-camptothecin, CAS No. 7689-03-4, Molecular weight: 34.36 g/mol, Aldrich product No. 36,563-7

5-(1-o-carboranyl)-pentanoic acid, F.W. 244.34 g/mol, Katchem, Prague, Czech Republic,

DL-2,3-diaminopropionic acid monohydrochloride, C3H8N2O2.HCl, CAS No. 54897-59-5, Acros Organics (New Jersey USA; Ceel Belgium) Product No. 204670050

4-[N-2,4-diamino-6-pteridinyl-methyl)-N-metylamino]-benzoic acid, hemihydrochloride dihydrate, CAS No. 19741-14-1, Aldrich product No. 86,155-3

2,6-dichlorobenzoyl chloride, CAS No. 225-102-4, Molecular weight: 209.46 g/mol, Lancaster product No. 8922

Diethylenetriaminepentaacetic dianhydride, CAS No. 23911-26-4, molecular weight: 357.32 g/mol, Aldrich product no. 28,402-5

DIPEA=N,N-Diisopropylethylamine, 2.0 M solution in N-methylpyrrolidone, Applied Biosystems Cat. No. 401517

DMAP; N-dimethylaminopyridine, CAS No. 1122-53-3, molecular weight: 122.17 g/mol, Fluka product no. 29224

Dota tris(t-Bu ester), Macrocyclics, Molecular weight: 572.74 g/mol

Doxorubicin hydrochloride, CAS No. 25316-40-9, molecular weight: 580.0 g/mol, Sigma Cat. No. D-1515

Fmoc-6-aminohexanoic acid (Fmoc-6-Ahx-OH), CAS No. 88574-06-5, Molecular Weight: 353.4 g/mol, Novabiochem Product No. 04-12-1111 A22837

Fmoc-L-Arg(Pbf)-OH, CAS No. 154445-77-9, Applied Biosystems Cat. No. GEN911097, Molecular Weight: 648.8 g/mol

Fmoc-Asp(2-phenylisopropyl ester)-OH, Molecular weight: 473.53 g/mol, Bachem Product No. B-2475.0005

Fmoc-L-Asn-OH, CAS No. 71089-16-7, Applied Biosystems, product no: GEN 911018

Fmoc-Gly Resin, Applied Biosystems Product No. 401421 0.65 mmol/g

Fmoc-Gly-OH, CAS No. 29022-11-5, Novabiochem Product No. 04-12-1001, Molecular Weight: 297.3 g/mol

Fmoc-L-Asn-OH, Applied Biosystems Product No. Gen 911018, Molecular weight: 354.40 g/mol

Fmoc-L-Arg(Pbf)-OH, CAS No. 154445-77-9, Applied Biosystems Product No. GEN911097, Molecular Weight: 648.8 g/mol

Fmoc-L-Cys(Trt)-OH, CAS No. 103213-32-7, Applied Biosystems Product No. GEN911027, Molecular Weight: 585.7 g/mol

Fmoc-L-Glu(OTMSEt)-ONa; N-2-Fmoc-glutamic acid 5-(2-trimethylsilylethyl) ester sodium salt, molecular weight: 468.60 g/mol, Novabiochem Cat. No. 04-12-1231

Fmoc-L-Glu(OtBu)-OH, CAS No. 71989-18-9, Applied Biosystems Product No. GEN911036, Molecular Weight: 425.5 g/mol

Fmoc-L-Leu-OH, CAS No. 35661-60-0, Applied Biosystems Product No. GEN911048, Molecular Weight: 353.4 g/mol

Fmoc-L-Lys(Fmoc)-OH, CAS No. 78081-87-5, Molecular weight: 590.7 g/mol, PerSeptive Biosystems (Hamburg, Germany) Product No. GEN911095

Fmoc-Lys(Mtt)-OH, Novabiochem Product No. 04-12-1137, Molecular Weight: 624.8 g/mol

Fmoc-Lys(Mtt) Resin, 0.68 mmol/g, Bachem Product No. D-2565.0005

Fmoc-L-Lys(tBoc)-OH, CAS No. 71989-26-9, Molecular Weight: 468.6 g/mol, Applied Biosystems Product No. GEN911051

Fmoc-D-Orn(Mtt)-OH; 2-N-Fmoc-5-N-(4-methyltrityl)-D-ornithine, molecular weight: 610.8 g/mol, Novabiochem Cat. No. 04-13-1012.

Fmoc-Ser(tBu) Resin, Applied Biosystems Product No. 401429, 0.64 mmol/g

Fmoc-L-Ser(tBu)-OH, CAS No. 71989-33-8, Perseptive Biosystems Product No. GEN911062, Molecular Weight: 383.4 g/mol

Fmoc-L-Tyr(tBu)-OH, CAS No. 71989-38-3, Molecular weight: 459.5 g/mol, Applied Biosystems Product No. GEN911068

HOAt=1-Hydroxy-7-azabenzotriazole, 0.5 M solution in DMF, Applied Biosystems Cat. No. 4330631

HBTU=2-(1H-benzotriazol-1-yl)-1,1,3,3-tetramethyluronium hexafluorophosphate, CAS No. [94790-37-1], Applied Biosystems Cat. No. 401091, molecular weight: 379.3 g/mol

HOBt=1-Hydroxybenzotriazole, 0.5 M solution in DMF, Applied Biosystems Cat. No. 400934

HMP Resin, loading capacity: 1.16 mmol/g (as reported by the producer of the commercial product), Applied Biosystems Cat. No. 400957

Iodine, CAS No. 7553-56-2, molecular weight: 253.81, Merck Art. No. 4760 4-nitrophenyl chloroformate, CAS No. 4693-46-1, Molecular weight: 201.57 g/mol, Fluka product No. 23240

Paclitaxel, from Tacsus yannesis, CAS No. 33069-62-4, Molecular weight: 853.9 g/mol, Sigma product No. T-1912

PYAOP=7-Azabenzotriazol-1-yloxytris(pyrrolidino)phosphonium hexafluorophosphate, CAS No. 156311-83-0, PE Biosystems Cat. No. GEN076531, Molecular Weight: 521.4 g/mol

PyBroP; Bromo-trispyrrolidinophosphonium hexafluorophosphate, CAS No. 132705-51-2, Molecular weight: 466.2 g/mol, Novabiochem product No. 01-62-0017

Rink amide MBHA resin, Loading 0.64 mmol/g, Novabiochem product No. 01-64-0037

Succinic anhydride, CAS No. 108-30-5, Molecular weight: 100.01 g/mol Fluka product No. 140089

List of Suppliers

Acros Organics, New Jersey USA; Ceel Belgium

Applied Biosystems, Warrington, WA1 4SR, United Kingdom

Bachem AG, Hauptstrasse 144, CH-4416 Bubendorf, Switzerland

Calbiochem-Novabiochem, CH-4448 Läufelfingen, Switzerland

Katchem, Prague, Czech Republic,

Lancaster, Morecambe, England

Fluka Chemie GmbH, Buchs, Switzerland

Macrocyclics, Dallas, Tex., USA

Merck KGaA, Darmstadt, Germany

PE Biosystems, Warrington, United Kingdom

Perseptive Biosystems, Warrington, United Kingdom/Hamburg Germany

Sigma Aldrich Chemie, Steinheim Germany

(also Riedel-deHaën)

Sigma-Genosys LTD, Pampisford, Cambridge, UK

Bio-Whittaker, Verviers, Belgium

Harlan Laboratories, Horst, The Netherlands

Genset SA, Paris, France

AmershamPharmacia Biotech, Uppsala, Sweden

Qiagen, Hilden, Germany

Terumo, Leuven, Belgium

Vector Laboratories, Burlingame, USA

### REFERENCES

Adams, G P, Schier R. Generating improved single-chain Fv molecules for tumor targeting. J Immunol Methods 1999; 231:249-60.
Arap, W., Pasqualini, R., and Ruoslahti, E. (1998). Chemotherapy targeted to tumor vasculature. Curr. Op. Oncol. 10: 560-565.
Auvinen, M, Laine, A., Paasinen-Sohns, A., Kangas, A., Saksela, O., Andersson, L. C., and Hölttä, E. (1997). Human ornithine decarboxylase-overproducing NIH3T3 cells induce rapidly growing, highly vascularized tumors in nude mice. Cancer Res. 57: 3016-25.
Bachem AG, SASRIN™, A review of its manyfold applications including many useful procedures, comlied by Mergier M., 2^{nd} revised and enlarged edition 1999, 1999 by BACHEM AG, CH-4416 Bubendorf, Switzerland.
Bachem 2001, Peptides and Biochemicals, Immunochemicals, The New 2001 BACHEM catalog, BACHEM AG, Hauptstrasse 144, CH-4416 Bubendorf Switzerland.
Beckman, G.; Beckman, L.; Ponten J. and Westermark B. (1971) G-6-PD and PGM phenotypes of 16 continuous human tumor cell lines. Evidence against cross-contamination and contamination by HeLa cells. Hum. Hered. 21: 238-241.
Biosystems Solutions, Issue 2—September 2001, p. 30. AB Applied Biosystems.
Chan, W. C. Bycroft, B. W., Evans, D. J. and White, P. D. A novel 4-aminobenzyl ester-based carboxy-protecting group for synthesis of atypical peptides by Fmoc-Bu^{t} solid-phase chemistry. (1995) J. Chem. Soc., Chem. Commun., 1995, p. 2209.
Ellerby, H. M., Arap, W., Ellerby, L. M., Kain, R., Andrusiak, R., Rio, G. D., Krajewski, S., Lombardo, C. R., Rao, R., Ruoslahti, E., Bredesen, D. E., and Pasqualini, R. 1(1999). Anti-cancer activity of targeted pro-apoptotic peptides. Nat. Med. 9:1032-1038.
Fluka Chemika, Peptide and Peptidomimetic Synthesis, Reagents for Drug Discovery, 2000 Fluka Chemie GmbH, Buchs, Fluka, Speciality Chemicals and Analytical Reagents.
Fogh J.; Fogh J M. and Orfeo T. (1977) One hundred and twenty-seven cutured human tumor cell lines producing tumors in nude mice. J. Natl. Cancer Inst. 59: 221-226.
Herndier B G, Werner A, Arnstein P, Abbey N W, Demartis F, Cohen R L, Shuman M A, Levy J A. (1994). Characterization of a human Kaposi's sarcoma cell line that induces angiogenic tumors in animals. AIDS 8:575-81.
Hidalgo, M., and Eckhardt, S. G. (2001). Development of matrix metalloproteinases inhibitors in cancer therapy. J. Natl. Cancer Inst. 93: 178-193.
Hirschmann, R., Yao, W., Arison, B., Maechler, L., Rosegay, A., Spengeler, P. A., and Smith, A. B. (1998), Synthesis of the first tricyclic homodetic peptide. Use of Coordinated Orthogonal Deprotection to Achieve Directed Ring Closure. Tetrahedron 54 (1998) 7179-7202.
Houghten, R. A., Pinilla C., Appel J. R., Blondelle S. E., Dooley C. T., Eichler J., Nefzi A., Ostresh J. M. Mixture-based synthetic combinatorial libraries. J. Med. Chem. 1999; 42:3743-78.
Mase K, Iijima T, Nakamura N, Takeuchi T, Onizuka M, Mitsui T, Noguchi M. Intrabrochial orthotopic propagation of human lung adenocarcinoma—characterizations on tumorigenicity, invasion and metastasis. Lung cancer 36 (3): 271-276, 2002.
Mergler, M. and Durieux, J. P., BACHEM AG, 2000 by BACHEM AG, CH-4416 Bubendorf, Switzerland.
Naknishi, H, and Kahn, M. (1996). Design of peptidomimetics. In: The practice of medical chemistry, pp. 571-590. Academic Press
Nargund, R. P., Patchett, A. A., Bach, M. A. Murphy, M. G., Smith, R. G. (1998) Peptidomimetic growth hormone secretagogues. Design considerations and therapeutic potential. J. Med. Chem. 1998; 41:3103-27.
Nicklin, S. A., White, S. J., Watkins, S. J., Hawkins, R. E., Baker, A. H. (2000). Selective targeting of gene transfer to vascular endothelial cells by use of peptides isolated by phage display. Circulation 102: 231-237.
Novabiochem # 1 for innovation. Novabiochem 2000 Catalog, Calbiochem-Novabiochem AG, Weidenmattweg 4 Läufelfingen, Switzerland, 2000. Peptide and Peptidomimetic Synthesis, Reagents for Drug Discovery, Fluka ChemieGmbH, Buchs, Switzerland, 2000
Prochiantz, A. (1996). Getting hydrophilic compounds into cells: lessons from homeopeptides. Curr. Op. Neurobiol. 6: 629-634.
Promega Notes Magazine, Promega Corporation, Number 74, InCELLect™ Cell-Permeable Peptides, 2000.
Protective Groups in Organic Synthesis, Third Edition, Theodora W. Greene, Peter G. M. Wuts, 1999, John Wiley & Sons, Inc. ISBN: 0-471-16019-9
The BACHEM Practise of SPPS, (2000) Tips and tricks from the experts at BACHEM, compiled by Mergier, M. and Durieux, J. P., BACHEM AG, 2000 by BACHEM AG, CH-4416 Bubendorf, Switzerland.
The Combinatorial Chemistry Catalog & Solid Phase Organic Chemistry (SPOC) Handbook, Novabiochem # 1 for innovation, Switzerland, Calbiochem-Novebiochem AG Weidenmattweg 4, CH-4448 Läufellingen, Switzerland, 1998-1999.
Welch, D. R., Bisi, J. E., Miller, B. E., Conaway, D., Seftor, E. A., Yohem, K. H. et al. (1991). Characterization of a highly invasive and spontaneously metastatic human malignant melanoma cell line. Int. J. Cancer 47: 227-237.
Yue, C., Thierry, J. and Potier, P. (1993) 2-phenyl isopropyl esters as carboxyl terminus protecting groups in the fast synthesis of peptide fragments, Tetrahedron Letters 34(2): 323-326.

1. A tumor targeting unit comprising a peptide sequence:
Cy-Rrₙ-Dd-Ee-Ff-Rrₘ-Cyy
or a pharmaceutically or physiologically acceptable salt thereof, wherein,
Dd-Ee-Ff is Aa-Bb-Cc or Cc-Bb-Aa, wherein
Aa, is isoleucine, leucine or tert-leucine, or a structural or functional analogue thereof,
Bb is arginine, homoarginine or canavanine, or a structural or functional analogue thereof;
Cc is serine or homoserine, or a structural or functional analogue thereof;
Rr are each, independently, any amino acid residue or a structural or functional analogue thereof;
n and m are, independently, 0, 1, or 2, and the sum of n and m does not exceed two; and,
Cy and Cyy are entities capable of forming a cyclic structure.

2. The tumor targeting unit according to claim 1, wherein the peptide is cyclic or forms part of a cyclic structure.

3. The tumor targeting unit according to claim 2, wherein the cyclic structure is formed through an amide, lactam or disulphide bond.

4. The tumor targeting unit according to claim 3, wherein one of Cy and Cyy is aspartic acid, glutamic acid or a structural or a functional analogue thereof, and the other is lysine, ornithine or a structural or functional analogue thereof.

5. The tumor targeting unit according to claim 3, wherein Cy and Cyy are cysteine or a structural or functional analogue thereof.

6. The tumor targeting unit according to claim 1, wherein Rr are any amino acid residues, except histidine or lysine.

7. The tumor targeting unit according to claim 6, wherein Rr is selected from the group consisting of glycine, arginine and structural or functional analogues thereof.

8. The tumor targeting unit according to claim 5, selected from the group consisting of CLRSC (SEQ ID NO. 1), CSRLC (SEQ ID NO. 2).

9. The tumor targeting unit according to claim 4, selected from the group consisting of DLRSK (SEQ ID NO. 3), DLRSGRK (SEQ ID NO. 4), DRGLRSK (SEQ ID NO. 5), OLRSE (SEQ ID NO. 6) and KLRSD (SEQ ID NO. 7).

10. The tumor targeting unit according to claim 1, wherein the unit is derivatized, activated, protected, resin bound or other support bound.

11. A tumor targeting agent comprising at least one targeting unit according to claim 1, directly or indirectly coupled to at least one effector unit.

12. The tumor targeting agent according to claim 11, wherein the effector unit is a directly or indirectly detectable agent or a therapeutic agent.

13. The tumor targeting agent according to claim 12, wherein the detectable agent comprises an affinity label, a fluorescent or luminescent label, a chelator, a metal complex, an enriched isotope, radioactive material or a paramagnetic substance.

14. The tumor targeting agent according to claim 13, wherein the detectable agent comprises a rare earth metal.

15. The tumor targeting agent according to claim 14, wherein the detectable agent comprises gadolinium.

16. The tumor targeting agent according to claim 12, wherein the therapeutic agent is selected from the group consisting of cytotoxic, cytostatic and radiation emitting substances.

17. The tumor targeting agent according to claim 16, wherein the therapeutic agent comprises doxorubicin, daunorubicin, methotrexate or boron.

18. The tumor targeting agent according to claim 11, further comprising an optional unit.

19. A diagnostic or pharmaceutical composition comprising at least one targeting unit according to claim 1.

20. A method for the preparation of a medicament for the treatment of cancer or cancer related diseases comprising using a targeting unit according to claim 1.

21. The method according to claim 20, wherein said cancer or cancer related disease is a solid tumor.

22. The method according to claim 21, wherein said solid tumor is selected from the group consisting of carcinoma, sarcoma, melanoma or metastases.

23. A method for treating cancer or cancer related diseases, comprising providing to a patient in need thereof a therapeutically effective amount of a pharmaceutical composition according to claim 19.

24. The method according to claim 23, wherein said cancer or cancer related disease is a solid tumor.

25. The method according to claim 24, wherein said solid tumor is selected from the group consisting of carcinoma, sarcoma, melanoma or metastases.

26. A diagnostic or pharmaceutical composition comprising at least one targeting agent according to claim 11.

27. A method for the preparation of a medicament for the treatment of cancer or cancer related diseases comprising using a targeting agent according to claim 11.

28. A method for treating cancer or cancer related diseases, comprising providing to a patient in need thereof a therapeutically effective amount of a pharmaceutical composition according to claim 26.
